(19) United States
(12) Patent Application Publication
(10) Pub. No.: US 2023/0137466 A1
(43) Pub. Date: May 4, 2023

(54) SYSTEM AND METHOD FOR ASSISTING FLOW OF A FLUID IN A VASCULAR SYSTEM OF A MAMMALIAN BODY

(71) Applicant: Puzzle Medical Devices Inc., Montreal (CA)

(72) Inventors: Gabriel Georges, Quebec (CA); Maxime Rochon, Quebec (CA)

(73) Assignee: Puzzle Medical Devices Inc., Montreal (CA)

(21) Appl. No.: 17/837,463

(22) Filed: Jun. 10, 2022

Related U.S. Application Data
(63) Continuation of application No. PCT/IB2020/061913, filed on Dec. 14, 2020.
(60) Provisional application No. 62/947,520, filed on Dec. 12, 2019.

(51) Int. Cl. A61M 60/139; A61M 60/32; A61M 60/33; A61M 60/295

(52) U.S. Cl. CPC A61M 60/139; A61M 60/32; A61M 60/33; A61M 60/295

(57) ABSTRACT

System for assisting vascular fluid flow body comprising: Fluid pump having an inlet for fluid in the vascular system to enter and an outlet for discharging pumped fluid into the vascular system at a fluid pump outlet pressure. Occluder placed into the vascular system at a location downstream of the outlet of the fluid pump. Occluder having a configuration in which the occluder occludes the vascular system and a configuration in which pumped is capable of flowing past the occluder. In use, the occluder being actuatable to alternate between the occluded configuration, in which pumped fluid accumulates in the vascular system becoming increasingly under pressure via elastic deformation of vessels, and the flow configuration, in which accumulated pumped fluid under pressure flows past the occluder at a greater pressure than the fluid pump outlet pressure, returning the elastically deformed vessels towards their original state.

### CROSS-REFERENCE

The present application is a continuation of International Application No. PCT/IB2020/061913, filed Dec. 14, 2020 (the “'913 PCT”), which claims priority to and the benefit of U.S. Provisional Patent Application No. 62/947,520, filed Dec. 12, 2019. The '913 PCT is related to International Patent Application Publication No. WO 2020/198765 A2, filed May 4, 2020 (the “WO '765 Publication”). The contents of each of the foregoing applications are incorporated herein by reference in their entirety for all purposes.

### FIELD

The present technology relates to system and methods for assisting flow of a fluid in a vascular system of a mammalian body.

### BACKGROUND

Healthy human adult cardiovascular circulatory systems carry approximately 5L of blood flow per minute. System pressures vary in relation to the heart's systole and diastole, resulting in systolic and diastolic pressures in the range of 120 and 80 mmHg, respectively. In patients with compromised hemodynamics, such as patients with severe systolic heart failure, cardiac output is decreased, resulting in relatively low flow through their circulatory system. Low flow conditions may result in life limiting symptoms such as dyspnea and vicious cycles such as cardiorenal syndrome due to reduced kidney perfusion. These conditions lead to life-limiting symptoms in patients and tremendous economic burden on the healthcare system.

A subset of patients with severe heart failure receive ventricular assist devices to increase their native blood flow. Traditionally, this refers to surgically implanted blood pumps with an apical inlet and an outlet anastomosed to the ascending aorta. Other percutaneous pumps can be positioned through the aortic valve and feature an intra-ventricular inlet and a supra-valvular outlet. Intra-aortic pumps such as those described in the WO '765 Publication can also be used.

These pumps typically offer continuous flow support in contrast to the native pulsatile flow. This results in normalization of blood flow through the system. However, native pulse pressure (the difference between systolic and diastolic pressures) is greatly reduced or lost. The loss of pulse pressure is believed to produce adverse events such as altered angiogenesis or ‘leaky vessels’ which result in cerebral and gastro-intestinal bleeding. Vital organs depending greatly on perfusion pressures for filtering of the blood, such as the kidneys and liver, may also be affected by this loss of pulse pressure. The art currently provides no optimal solution to simulate this pulse pressure differential when such pumps are being used.

Therefore, there is a need for a system capable of providing at least some pulse pressure differential in hemodynamically supported patients to reduce or overcome (if possible, depending on the circumstances) at least some of the above-described problems.

### SUMMARY

It is an object of the present technology to ameliorate at least some of the inconveniences present in the prior art.

It is another object of the present technology to provide a system for providing at least some pulse pressure differential in hemodynamically supported patients.

At a high level, the present technology exploits the native elasticity of the blood vessels of the vasculature to store blood flow from a pump under pressure by periodically blocking off the blood vessel(s) downstream of the pump (letting the pumped blood accumulate between the pump outlet and the blockage, stretching out the blood vessel) and unblocking the blood vessel(s) (letting the blood flow downstream from the former blockage, under pressure, as the stretched out blood vessels return to their “normal” unstretched out state.)

In the medical arts, a blocked blood vessel is generally referred to as “occluded” or “obstructed” the blood vessel. Similarly, a blockage in a blood vessel is generally referred to as an “occlusion” or an “obstruction”. In the context of the description of the present technology, “occluded” and “occlusion” will generally be used throughout, for the purpose of consistency and ease of reading. No distinction between the term “occlusion” and the terms “block” and “obstruct” and their various forms is intended in the present context.

In one aspect, some embodiments of the present technology provide a system for assisting a flow of a fluid in a vascular system of a mammalian body, the vascular system having a plurality of vessels. The fluid flow assist system comprises: A fluid pump structured and arranged to be placed in fluid communication with the fluid in the vascular system. The fluid pump has an inlet for fluid in the vascular system to enter the pump and an outlet for discharging pumped fluid into the vascular system at a fluid pump outlet pressure. An occluder, which is structured and dimensioned to be placed into the vascular system at a location downstream of the outlet of the fluid pump. The occluder has an occluded configuration in which the occluder occludes the vascular system such that pumped fluid flowing in the vascular system from the outlet of the fluid pump towards the occluder is blocked from flowing past the occluder. The occluder has a flow configuration in which pumped fluid flowing in the vascular system from the outlet of the fluid pump towards the occluder is capable of flowing past the occluder. An actuator is operatively connected to the occluder for actuating the occluder between the occluded configuration and the flow configuration. In use, the occluder being actuatable to alternate between (at least) two configurations. An occluded configuration, in which pumped fluid accumulates in the vascular system between the outlet of the fluid pump and the occluder becoming increasingly under pressure via elastic deformation of vessels in the vascular system. And, a flow configuration, in which accumulated pumped fluid under pressure flows past the occluder at a greater pressure than the fluid pump outlet pressure, returning the elastically deformed vessels towards their original state.

In the context of the present technology, an “occluder” is any device or combination of devices that, as properly implanted in vivo, has the ability to (materially, and preferably completely) occlude a blood vessel in to which it has been implanted. No particular structure is required as long as the functions described herein can be carried out and that structure (whatever it may be) is capable of operating inside the vasculature of a body into which it would be implanted or otherwise within its operating environment. Thus, an occluder of the present technology may be a mechanical, electromechanical, or electronic structure, for example.

As is set forth above, an occluder of the present technology has an “occluded configuration” and a “flow configuration”. These configurations are operational configurations, and any structure that has such operational configurations and is capable of interconverting between the two at an appropriate rate is within the scope of the present technology. In the occluded configuration, although in many embodiments it will be most beneficial if this is the case, it is not required that there be zero fluid flow past the occluder. As long as the device is capable of providing the benefits described herein with respect to fluid pressure differential, a small flow past the occluder, when in the occluded configuration, is permitted. Similarly, in the flow configuration, while in most embodiments the greatest amount of flow would be beneficial, it is not required that the fluid flow past the occluder would have the same flow rate as it would have had natively (had there been no fluid pump at all) or had the occluder not have been present (but a fluid pump being present).

Any type of actuator (be it simple or complex, conventional or future) that is compatible with the occluder (in the sense that the actuator is capable of interconverting the occluder between the occluded configuration and the flow configuration (or letting the occluder convert between the two), as long as the actuator is not otherwise incompatible with being implanted in and operating in the environment of the vasculature of the human body, is within the scope of the present invention. No particular method of operation of an otherwise suitable actuator is required; an actuator of the present technology may thus be, for example, mechanical, electromechanical, or electronic.

Further, as a skilled addressee would understand, an actuator and an occluder of the present technology, need not be separate physical devices or structure. They may be combined into one physical device, competent or structure, as the case may be in any particular embodiment or implementation.

Finally, any fluid pump (be it conventional or future) that is not otherwise incompatible with use with an occluder (for whatever reason it may be) is within the scope of the present technology. While the developer of the present technology foresees that a greater benefit to the patient into which the present system will be implanted is through the use of an intravascular transcatheterly implanted fluid pump (in combination with an occluder), other types of fluid pumps are within the scope of the present technology. As a non-limiting example, the fluid pumps (being ventricular assist devices (“VAD's”)) described and claimed in the WO '765 Publication are within the scope of the present technology, as are at least some of the other types of VAD's (for example) described within the background section of the WO '765 Publication).

Thus, in some embodiments the fluid pump is an intravascular pump. In some embodiments the fluid pump is an intraventricular pump. In some embodiments the fluid pump is a transaortic pump. In some embodiments the fluid pump is an extracorporeal pump.

In some embodiments, the fluid pump is (and is operated as) a continuous flow pump.

Further, in some embodiments the fluid pump, the occluder and the actuator are a unitary structure, be it manufactured as a single unit or manufactured as separate units and joined together prior to implantation or operation (as the case may be). In some embodiments the fluid pump, the occluder and the actuator are separate structures (irrespective of whether the occluder and the actuator are themselves unitary or separate structures).

In some embodiments the system further comprises a controller operatively connected to the actuator for controlling the actuator. Any type of controller otherwise compatible with the other components of the system (and not incompatible with the operating environment of the system) is within the scope of the present technology. In some embodiments, the controller is an electronic controller such as a microchip, integrated circuit, etc. The controller may be a separate component or may be incorporated into one of the other components of the system; e.g., the controller and the actuator may be a unitary structure. In some embodiments a single controller is used to control both the actuator and the fluid pump. In some embodiments the controller includes a timer, any appropriate conventional timer may be used. The controller need not be electronic, and in some embodiments the controller is a mechanical controller.

In some embodiments, the system further comprises a power source. In some such embodiments the power source is extravascular, and the system further comprises a catheter allowing for electrical interconnection of the power source and at least the actuator. In some embodiments the power source is intravascular, and it may be a separate component or incorporated into one of the other components of the system. In some embodiments, the power source is the same power source as a power source used to power the fluid pump. In some embodiments the power source is a power source different from the one used to power the fluid pump. In some embodiments the power source is a rechargeable battery.

In some embodiments the system further comprises a pressure sensor that is structured, arranged, and positioned within the system to be capable of measuring the pressure of the blood in the vasculature fluidly between the outlet of the fluid pump and the occluder. The pressure sensor may be any type of appropriate pressure sensor capable of carrying out the function described herein. In some embodiments the pressure sensor is an electronic or electromechanical pressure sensor. In some such embodiments the pressure sensor is in electronic communication with the controller and outputs a signal indicative of the pressure usable by the controller. In some embodiments the pressure sensor is a mechanical pressure sensor.

In some embodiments the system further comprises at least one sensor operatively connected to the controller for providing data relevant to control of the actuator and thereby the configuration of the occluder. In some embodiments the data is relevant for at least electrocardiogram data analysis. In some embodiments the data is relevant for at least vessel fluid pressure curve analysis. In some embodiments the data is relevant for at least pulse oximetry curve analysis. In some embodiments the data is relevant for at least soundwave analysis. In some embodiments the data is related to continuous monitoring of a physiologic parameter. Any appropriate sensor may be used.

In some embodiments, the at least one sensor is a position sensor for determining the configuration (e.g., the position) of the occluder.

In some embodiments the occluder includes an operationally static element and an operationally movable element. In some such embodiments, the operationally movable element is translationally movable with respect to the operationally static element between two relative positions; a first relative position in which the occluder is in the occluded configuration and a second relative position in which the occluder is in the flow configuration.

In some such embodiments, when the operationally movable element and the operationally static element are in the first relative position, fluid flow ports on each of the elements are completely nonaligned, preventing fluid communication between the ports. Further, when the operationally movable element and the operationally static element are the in second relative position, fluid flow ports on each of the elements are at least in incomplete alignment, allowing fluid communication between the ports.

In some such embodiments, the operationally movable element is rotationally movable with respect to the operationally static element between first relative position in which the occluder is in the occluded configuration and a second relative position in which the occluder is in the flow configuration. In some such embodiments, when the operationally movable element and the operationally static element are in the first relative position, fluid flow ports on each of the elements are completely nonaligned, preventing fluid communication between the ports. In some such embodiments, when the operationally movable element and the operationally static element are the in second relative position, fluid flow ports on each of the elements are at least in incomplete alignment, allowing fluid communication between the ports.

In some embodiments the occluder includes a valve. The valve may be any suitable valve, such as a mechanical valve, electromechanical valve, an electronic valve or biological valve.

In some embodiments the occluder includes at least one element transformable between a first state in which the occluder is in the occluded configuration and a second state in which the occluder is in the flow configuration. In some such embodiments, the element is transformable between the first state and the second state via a change in shape. In some such embodiments, the element is transformable between the first state and the second state via a change in dimension. In some embodiments the element consists essential of an elastically deformable metal alloy. In some embodiments the element consists essential of nitinol.

In some embodiments the occluder includes a balloon sized and shaped to be inflated within the vascular system of the mammalian body.

In some such embodiments the occluder is sized such that a maximum diameter of the element (see the previous paragraph) and the anchor is less than the diameter of the location. This may be the case, for example, when it is not desired to totally occlude blood from flowing past the occluder when the occluder is in the occluded configuration. Such may be the case when the occluder is upstream from the carotid arteries, for example.

In some such embodiments the occluder is sized such that a maximum diameter of the element and the anchor is equal to the diameter of the location.

In some such embodiments, the occluder is sized such that a maximum diameter of the element and the anchor is greater than the diameter of the location. This may be the case, for example, when the occluder includes a balloon, which, when inflated will likely be greater than the diameter of the location in order to remain firmly in place and to maintain occlusion.

In some embodiments the occluder includes an anchor for anchoring the occluder in place with respect to the vascular system.

In some embodiments the entire system is intravascular. In some embodiments the entire system, with the exception of the power source (and its associated wiring, if any), is intravascular. In some embodiments the entire system, with the exception of the power source and the controller (and their associated wiring, if any), is intravascular.

In some embodiments the fluid is blood. In some such embodiments the flow of the fluid is a native cardiac output.

In some embodiments, the mammalian body is a human body. In other embodiments, the mammalian body is a non-human animal body (i.e., the system is for veterinary use).

In another aspect, some embodiments of the present technology provide a system for assisting a flow of a fluid in a vascular system of a mammalian body, the vascular system having a plurality of vessels. The fluid flow assist system comprises: A fluid pump that is structured and arranged to be placed in fluid communication with the fluid in the vascular system. The fluid pump has an inlet for fluid in the vascular system to enter the pump and an outlet for discharging pumped fluid to the vascular system at a fluid pump outlet pressure. A first occluder is structured and dimensioned to be placed into a first branch of the vascular system at a location downstream of the outlet of the fluid pump. The first occluder has an occluded configuration in which the first occluder occludes the vascular system such that pumped fluid flowing in the vascular system from the outlet of the fluid pump towards the first occluder is blocked from flowing past the first occluder. The first occluder has a flow configuration in which pumped fluid flowing in the vascular system from the outlet of the fluid pump towards the first occluder is capable of flowing past the first occluder. A second occluder is structured and dimensioned to be placed into a second branch of the vascular system at a location downstream of the outlet of the fluid pump. The second occluder has an occluded configuration in which the second occluder occludes the vascular system such that pumped fluid flowing in the vascular system from the outlet of the fluid pump towards the second occluder is blocked from flowing past the second occluder. The second occluder has a flow configuration in which pumped fluid flowing in the vascular system from the outlet of the fluid pump towards the second occluder is capable of flowing past the second occluder. At least one actuator is operatively connected to the first occluder and to the second occluder for actuating the first occluder between the occluded configuration and the flow configuration and the second occluder between the occluded configuration and the flow configuration.

In use, the first occluder and the second occluder being actuatable to alternate between (at least) two states. A first state in which the first occluder is in the occluded configuration and the second occluder is in the occluded configuration. Pumped fluid accumulates in the vascular system between the outlet of the fluid pump, the first occluder and the second occluder, becoming increasingly under pressure via elastic deformation of vessels in the vascular system. And, a second state in which at least one of the first occluder is in the flow configuration and the second occluder is in the flow configuration. Accumulated pumped fluid under pressure flows past the at least one of the first occluder in the flow configuration and the second occluder in the flow configuration at a greater pressure than the fluid pump outlet pressure, returning elastically deformed vessels towards their original state.

In some embodiments in the second state only one of the first occluder is in the flow configuration and the second occluder is in the flow configuration. In some embodiments in the second state both the first occluder is in the flow configuration and the second occluder is in the flow configuration. In some embodiments in the second state both the first occluder is in the flow configuration and the second occluder is in the flow configuration simultaneously. In some embodiments in the second state both the first occluder is in the flow configuration and the second occluder is in the flow configuration sequentially

In this manner, native or augmented fluid (blood) flow may be preferentially oriented to specific organs of the body, for example, in patients receiving continuous flow hemodynamic support.

In some embodiments the at least one actuator is a first actuator and a second actuator. The first actuator is operatively connected to the first occluder for actuating the first occluder between the occluded configuration and the flow configuration. The second actuator is operatively connected to the second occluder for actuating the second occluder between the occluded configuration and the flow configuration.

In some embodiments the system further comprises a controller operatively connected to the first actuator for controlling the first actuator and to the second actuator for controlling the second actuator.

In some embodiments the system further comprises at least one sensor operatively connected to the controller for providing data relevant to control of at least one of the first actuator and the second actuator and thereby the state of the first occluder and the second occluder. In some such embodiments the at least one sensor is a first sensor and a second sensor. The first sensor is for providing data relevant to control of the first actuator and thereby the configuration of the first occluder. The second sensor is for providing data relevant to control of the second actuator and thereby the configuration of the second occluder.

In some embodiments the fluid is blood. In some such embodiments the flow of the fluid is a native cardiac output.

In some embodiments the mammalian body is a human body.

In yet another aspect, implementations of the present technology a method of assisting a flow of a fluid in a vascular system of a mammalian body, the vascular system having a plurality of vessels. The fluid flow assist method comprises:

(A) One of:
(i) surgically fluidly connecting an inlet of a fluid pump at a fluid-pump-inlet location within the vascular system of the body, and surgically fluidly connecting an outlet of the fluid pump at a fluid-pump-outlet location within the vascular system of the body, the fluid-pump outlet location being downstream from the fluid-pump-inlet location;
or
(ii) transcatheterly implanting a fluid pump within the vascular system of the body, the fluid pump having an inlet at a fluid-pump-inlet location and an outlet at a fluid-pump-outlet location within the vasculature.

In the context of the present technology, “surgically” should be understood to encompass traditional “open surgical” techniques whereas “transcatheterly” encompasses modern “minimally invasive” transcatheter techniques.

Thus, when implanting a traditional extravascular/extracorporeal (apical) LVAD for example, the inlet of the fluid pump is surgically attached to the left ventricle and the blood flow out of the heart into the external LVAD. By contrast, in an intravascular VLAD, the entire pump, including the inlet and outlet, is within the vasculature (having been transcatheterly implanted). Thus, there is no opening having been made in the vasculature that is in fluid communication with the pump inlet and through which blood flows into the pump.

Similar to the case with the inlet, the outlet may fluidly communicate with an opening having been in the vasculature for that purpose and through which blood flows out from the pump, in the case of an extracorporeal pump. Alternatively, in an intravascular blood pump (for example), the pump outlet simply outlets pumped blood into the vasculature (there is no opening in the vasculature having been made for this purpose).

(B) Implanting an occluder within the vascular system of the body at an occluder location, the occluder location being downstream from the fluid-pump-outlet location.

In some implementations, implanting an occluder is surgically implanting an occluder. In some implementations, implanting an occluder is transcatheterly implanting an occluder.

In some implementations there may be only a single fluid pathway between the fluid-pump-outlet location and the occluder location, but this is not required to be the case in every implementation. In implementations where there is an unoccluded branch of the vasculature in the fluid pathway from the fluid-pump outlet location to the occluder location, the pressure difference resulting from the use of the system will likely be less than it would have been had there been no unoccluded branch of the vasculature in the fluid pathway. In most cases, the more unoccluded branches there off the pathway are the more the pressure difference will be attenuated, but there will likely still be an effect.

The occluder has an occluded configuration in which the occluder occludes the vascular system such that pumped fluid flowing in the vascular system from the outlet of the fluid pump towards the occluder is blocked from flowing past the occluder. The occluder has a flow configuration in which pumped fluid flowing in the vascular system from the outlet of the fluid pump towards the occluder is capable of flowing past the occluder.

(C) Operating the fluid pump to discharge fluid from the outlet of the pump into the vascular system at a fluid pump outlet pressure.

(D) Causing the occluder to assume occluded configuration causing pumped fluid to accumulate in the vascular system between the fluid-pump-outlet location and the occluder location. The accumulated pumped fluid becomes increasingly under pressure via elastic deformation of vessels in the vascular system.

(E) Causing the occluder to assume the flow configuration causing the accumulated pumped fluid under pressure to flow past the occluder at a greater pressure than the fluid pump outlet pressure. The elastically deformed vessels return towards their original state.

In some implementations the method further comprises repeating (D) to (E).

In some implementations operating the fluid pump is continuously operating the fluid pump.

In some implementations (A) includes transcatheterly implanting the fluid pump within the vascular system endovascularly. In some implementations (A) includes transcatheterly implanting the fluid pump intraventricularly or transaorticly.

In some implementations the occluder location is in the ascending aorta of the body, to increase coronary perfusion.

In some implementations the occluder location is in the proximal descending aorta of the body, to increase carotid artery and coronary perfusion.

In some implementations the occluder location is in axillary and iliac arteries of the body bilaterally, to increase carotid and visceral artery perfusion.

In some implementations the occluder location is in the descending aorta of the body in an infra-renal location, to increase kidney perfusion.

In some implementations the fluid pump is surgically implanted at location upstream of the renal arteries and the occluder location is downstream of the renal arteries, to increase at least one of renal artery perfusion pressure and pulse pressure.

In some implementations the fluid pump is surgically implanted at location upstream of the coeliac trunk and the occluder location is downstream of the superior mesenteric artery, to increase at least one of splanchnic artery perfusion pressure and pulse pressure.

In some implementations the fluid pump is surgically implanted at location upstream of the renal arteries and the occluder location is in the downstream of the renal arteries, to increase at least one of renal artery perfusion pressure and pulse pressure.

In some implementations the fluid pump is an intra-caval pump for right heart support.

In some implementations the fluid pump is surgically implanted in the inferior vena cava and the occluder location is in the superior vena cava, to increase right heart filling.

In some implementations the fluid pump is surgically implanted upstream of the renal veins in the inferior vena cava and the occluder location is downstream of the renal articles in the inferior vena cava, to increase venous renal decongestion.

In some implementations the method further comprises, after (B) and prior to (C), anchoring the occluder within the vascular system of the body at the occluder location. Any conventional anchoring system can be used. As a non-limiting example, the anchoring system described in U.S. Provisional Patent Application No. 62/957,115, filed Jan. 3, 2020, entitled “Lumen Wall Anchor for Use in Maintaining an Intralumenal Device Within a Mammalian Body Conduit” could be used. The entirety of the contents of that application are incorporated herein by reference.

In some implementations the method further comprises, after (B) and prior to (C), providing power to the fluid pump and an actuator operatively connected to the occluder.

In some implementations the method further comprises, prior to (D), continuously monitoring a physiological parameter of the body. And also, at least one of: (D) only when the monitored physically parameter exceeds a first predetermined threshold value, and (E) only when the monitored physically parameter exceeds a second predetermined threshold value.

In some implementations the method further comprises, at least one of: (D) only after a first predetermined period of time has elapsed. And, (E) only when a second predetermined period of time has elapsed.

In some implementations the fluid is blood. In some such implementations the flow of the fluid is a native cardiac output.

In some implementations the mammalian body is a human body.

In still another aspect implementations of the present technology provide a method of assisting a flow of a fluid in a vascular system of a mammalian body, the vascular system having a plurality of vessel. The fluid flow assist method comprises:

(A) One of (i) surgically fluidly connecting an inlet of a fluid pump at a fluid-pump-inlet location within the vascular system of the body, and surgically fluidly connecting an outlet of the fluid pump at a fluid-pump-outlet location within the vascular system of the body, the fluid-pump outlet location being downstream from the fluid-pump-inlet location; and (ii) transcatheterly implanting a fluid pump within the vascular system of the body, the fluid pump having an inlet at a fluid-pump-inlet location and an outlet at a fluid-pump-outlet location within the vasculature;

(B) Implanting a first occluder within the vascular system of the body at a first-occluder location. The first-occluder location is downstream from the fluid-pump outlet location. There is a first fluid pathway between the fluid-pump-outlet location and the first occluder location. The first occluder has an occluded configuration in which the first occluder occludes the vascular system such that pumped fluid flowing in the vascular system from the outlet of the fluid pump towards the first occluder is blocked from flowing past the first occluder. The first occluder has a flow configuration in which pumped fluid flowing in the vascular system from the outlet of the fluid pump towards the first occluder is capable of flowing past the first occluder. As was the case with other implementations described hereinabove, in some implementations the first occluder is surgically implanted; in some implementations the first occluder is transcatheterly implanted.

(C) Implanting a second occluder within the vascular system of the body at a second-occluder location. The second-occluder location is downstream from the fluid-pump outlet location. There is a second fluid pathway between the fluid-pump-outlet location and the second-occluder location. The second occluder has an occluded configuration in which the second occluder occludes the vascular system such that pumped fluid flowing in the vascular system from the outlet of the fluid pump towards the second occluder is blocked from flowing past the second occluder. The second occluder has a flow configuration in which pumped fluid flowing in the vascular system from the outlet of the fluid pump towards the second occluder is capable of flowing past the second occluder. As was the case with other implementations described hereinabove, in some implementations the second occluder is surgically implanted; in some implementations the second occluder is transcatheterly implanted.

(D) Operating the fluid pump to discharge fluid from the outlet of the pump into the vascular system at a fluid pump outlet pressure. As was the case with other implementations described hereinabove, in some implementations operating the fluid pump is continuously operating the fluid pump.

(E) Causing the first occluder to assume the occluded configuration and the second occluder to assume the occluded configuration causing pumped fluid to accumulate in the vascular system between the fluid-pump-outlet location, the first-occluder location and the second-occluder location. The accumulated pumped fluid becomes increasingly under pressure via elastic deformation of vessels in the vascular system.

(F) Causing at least one of the first occluder to assume the flow configuration and the second occluder to assume the flow configuration causing the accumulated pumped fluid under pressure to flow past the at least one of the first occluder and the second occluder at a greater pressure than the fluid pump outlet pressure. The elastically deformed vessels return towards their original state.

In some implementations the method further comprises repeating (E) to (F).

In some implementations, (F) is simultaneously causing the first occluder to assume the flow configuration and the second occluder to assume the flow configuration prior to repeating (E) to (F). In some implementations, (F) is sequentially causing the first occluder to assume the flow configuration and the second occluder to assume the flow configuration prior to repeating (E) to (F). In some implementations (F) is causing only one of the first occluder to assume the flow configuration and the second occluder to assume the flow configuration prior to repeating (E) to (F).

In some implementations the fluid is blood. In some such implementations the flow of the fluid is a native cardiac output.

In some implementations, there is no other fluid pathway other than the first fluid pathway and the second fluid between the fluid-pump-outlet location, the first-occluder location and the second-occluder location.

In some implementations the mammalian body is a human body.

In still yet another aspect, implementations of the present technology provide a method of assisting a flow of a fluid in a vascular system of a mammalian body, the vascular system having a plurality of vessels. The fluid flow assist method comprises: (A) operating a fluid pump to discharge fluid into the vascular system at a fluid pump outlet pressure. The fluid pump has an inlet having been surgically fluidly connected to the vascular system at a fluid-pump-inlet location. The fluid pump has an outlet having been surgically fluidly connected to the vascular system at a fluid-pump-outlet location. The fluid-pump outlet location is downstream from the fluid-pump-inlet location. (B) Causing an occluder (having been surgically implanted with the vascular system of the body at an occluder location, the occluder location being downstream from the fluid-pump outlet location, to assume an occluded configuration in which the occluder occludes the vascular system such that pumped fluid flowing in the vascular system from the outlet of the fluid pump towards the occluder is blocked from flowing past the occluder. Thus, causing pumped fluid to accumulate in the vascular system between the fluid-pump-outlet location and the occluder location. The accumulated pumped fluid becomes increasingly under pressure via elastic deformation of vessels in the vascular system. (C) Causing the occluder to assume a flow configuration in which pumped fluid flowing in the vascular system from the outlet of the fluid pump towards the occluder is capable of flowing past the occluder. Thus, causing the accumulated pumped fluid under pressure to flow past the occluder at a greater pressure than the fluid pump outlet pressure. The elastically deformed vessels return towards their original state.

In some implementations the method further comprises repeating (B) to (C).

In some implementations operating the fluid pump is continuously operating the fluid pump.

In some implementations the fluid pump has been surgically implanted within the vascular system percutaneously. In some implementations the fluid pump has been surgically implanted within the vascular system endovascularly via a catheter. In some implementations the fluid pump has been intraventricularly surgically implanted.

In some implementations the fluid is blood. In some such implementations the flow of the fluid is a native cardiac output.

In some implementations here is only a single fluid pathway between the fluid-pump-outlet location and the occluder location.

In some implementations the mammalian body is a human body.

### General

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first device” and “third device” is not intended to imply any particular order, type, chronology, hierarchy or ranking (for example) of/between the devices, nor is their use (by itself) intended imply that any “second device” must necessarily exist in any given situation. Further, as may be discussed herein in other contexts, reference to a “first” element and a “second” element does not preclude the two elements from being the same actual real-world structure.

In the context of the present specification, the word “embodiment(s)” is generally used when referring to physical realizations of the present technology and the word “implementations” is generally used when referring to methods that are encompassed within the present technology (which generally involve also physical realizations of the present technology). The use of these different terms is not intended to be limiting of or definitive of the scope of the present technology. These different terms have simply been used to allow the reader to better situate themselves when reading the present lengthy specification.

Embodiments and implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a schematic view of the human cardiovascular anatomy being a first implementation of the present technology where an occluder is positioned in the ascending aorta to increase coronary perfusion.

FIG. 2 illustrates a schematic view of the human cardiovascular anatomy being a second implementation of the present technology where an occluder is positioned in the proximal descending aorta to increase carotid artery and coronary perfusion.

FIG. 3 illustrates a schematic view of the human cardiovascular anatomy being a third implementation of the present technology where an occluder is positioned in the descending aorta in a supra-renal position to increase visceral artery perfusion.

FIG. 4 illustrates a schematic view of the human cardiovascular anatomy being a fourth implementation of the present technology where an occluder is positioned in the descending aorta in an infra-renal position to increase kidney perfusion.

FIG. 5 illustrates a schematic view of the human cardiovascular anatomy being a fifth implementation of the present technology where occluders are positioned in the axillary and iliac arteries bilaterally to increase carotid and visceral artery perfusion.

FIG. 6 illustrates a schematic view of the human cardiovascular anatomy being a sixth implementation of the present technology where an intra-aortic pump is positioned above the renal arteries and an occluder is positioned below the renal arteries to increase renal artery perfusion pressures or pulse pressures.

FIG. 7 illustrates a schematic view of the human cardiovascular anatomy being a seventh implementation of the present technology where an intra-aortic pump is positioned above the coeliac trunk and an occluder is positioned below the superior mesenteric artery to increase splanchnic artery perfusion pressures or pulse pressures.

FIG. 8 illustrates a schematic view of the human cardiovascular anatomy being an eight implementation of the present technology wherein an intra-caval pump is positioned in the inferior vena cava and an occluder is positioned in the superior vena cava to increase right heart filling.

FIGS. 9A-9G show a first embodiment of an occluder of the present technology. This first embodiment has two sub-embodiments. FIG. 9A is an isometric view of the device taken from a distal point of view when closed (e.g., in a flow configuration) (first sub-embodiment). FIG. 9B is a plan view of the device when closed (e.g., in a flow configuration) (first sub-embodiment). FIG. 9C is a plan view of the device when opened (e.g., in an occluded configuration) (second sub-embodiment). FIG. 9D is close up plan view of the device as shown in FIG. 9C (second sub-embodiment). FIG. 9E is an isometric view of the device when opened taken from a distal point of view (e.g., in an occluded configuration) (first-sub embodiment). FIG. 9F is a plan view of the device taken from an opposite side to that shown in FIGS. 9C and 9D (first sub-embodiment). FIG. 9G is an isometric view of the device when opened taken from a proximal point of view (e.g., in an occluded configuration) (first-sub embodiment).

FIGS. 10A-F show a second embodiment of an occluder of the present technology. FIG. 10A is an isometric view of the device taken from a distal point of view when in a closed configuration (e.g., in an occluded configuration). FIG. 10B is a plan view of the device when in a closed configuration (e.g., in an occluded configuration). FIG. 10C is an isometric view of the device taken from a distal point of view when in an opened configuration (e.g., in a flow configuration). FIG. 10D is a plan view of the device when in an opened configuration (e.g., in a flow configuration). FIG. 10E is an isometric view of the device taken from a proximal point of view when in a closed configuration (e.g., in an occluded configuration). FIG. 10F is an isometric view of the device taken from a proximal point of view when in an opened configuration (e.g., in a flow configuration).

FIGS. 11A-11H show a third embodiment of an occluder of the present technology. FIG. 11A is an isometric view of the device taken from a distal point of view when in a closed configuration (e.g., in an occluded configuration). FIG. 11B is a plan view of the device when in a closed configuration (e.g., in an occluded configuration). FIG. 11C is an isometric view of the device taken from a distal point of view when in an opened configuration (e.g., in a flow configuration). FIG. 11D is a plan view of the device when in an opened configuration (e.g., in a flow configuration.). FIG. 11E is a first close-up view of the valve of the device taken from a distal point of view when the device is in a closed configuration (e.g., in an occluded configuration). FIG. 11F is a second close-up view of the valve of the device when the device is in a closed configuration (e.g., in an occluded configuration), showing the pivoting mechanism. FIG. 11G is an isometric view of the device taken from a proximal point of view when in a closed configuration (e.g., in an occluded configuration). FIG. 11H is an isometric view of the device taken from a proximal point of view when in an opened configuration (e.g., in a flow configuration).

### DETAILED DESCRIPTION OF SOME EMBODIMENTS

In the figures there are shown various schematics of the human cardiovascular system in which systems of the present technology have been implemented. It is to be expressly understood that the various schematics are merely some implementations of the present technology. Thus, the description thereof that follows is intended to be only a description of illustrative examples of the present technology. This description is not intended to define the scope or set forth the bounds of the present technology. In some cases, what are believed to be helpful examples of modifications these schematics may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the schematics may provide in certain instances simple implementations of the present technology, and that where such is the case they have been presented in this manner as an aid to understanding. As persons skilled in the art would understand, various implementations of the present technology may be of a greater complexity. Finally, being “schematics”, the figures showing schematics are not intended to show in complete detail either the implementations of the present technology or the human cardiovascular system. They are intended to be representations only, addressed to persons of skill in the art.

### Description of Relevant Human Cardiovascular Anatomy

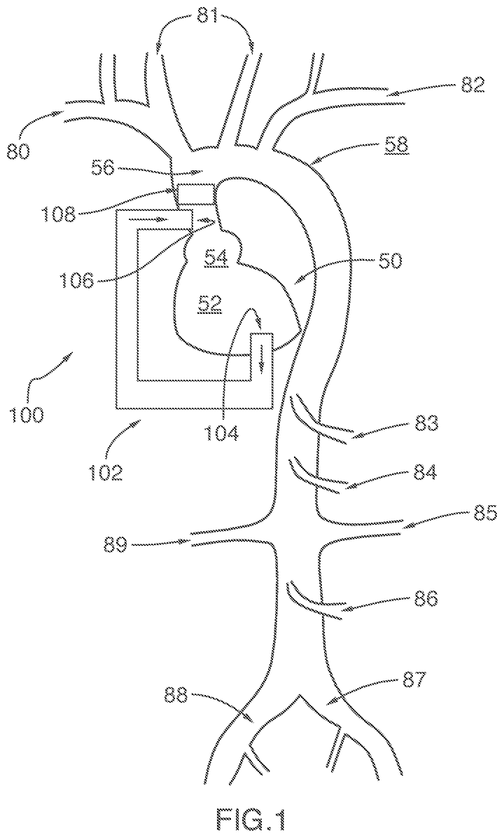

In FIGS. 1 to 7, the following parts of the human cardiovascular anatomy are shown and referenced: right axillary artery 80, carotid arteries 81, left axillary artery 82, coeliac trunk 83, superior mesenteric artery 84, left renal artery 85, inferior mesenteric artery 86, left common iliac artery 87, right common iliac artery 88, and the right renal artery 89.

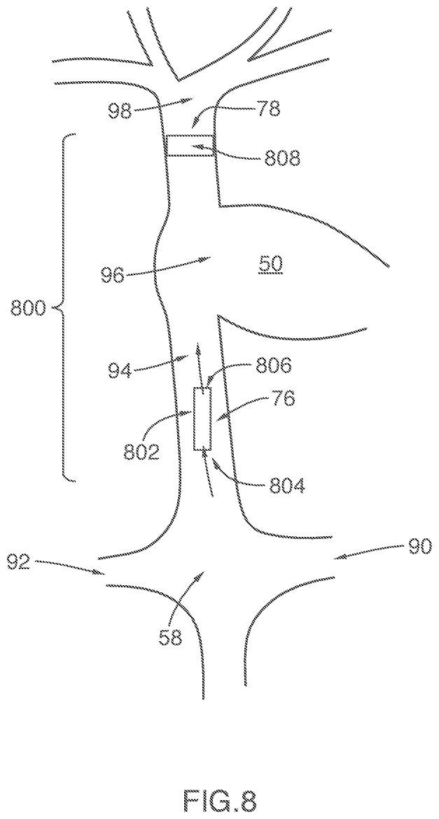

In FIG. 8, the following parts of the human cardiovascular anatomy are shown and referenced: left renal vein 90, right renal vein 92, inferior vena cava 94, right auricle 96 (of heart 50), and superior vena cava 98.

### First Implementation

Referring to FIG. 1, there is a shown a first implementation of the present technology 100. In this implementation there is a single fluid pump 102, being a conventional left ventricle assist device (LVAD) (an “apical pump”), which has been surgically implanted using conventional techniques. The LVAD 102 has a blood flow inlet 104 surgically fluidly connected to the left ventricle 52 of the heart 50. The left auricle 54 of the heart 50 is also shown in FIG. 1. The LVAD 102 has a blood flow outlet 106 surgically fluidly connected to the ascending aorta 56. A single occluder 108 (the structure of which is discussed hereinbelow) has been surgically transcatheterly implanted in the ascending aorta 56. (Transcatheter implantations generally are described in the WO '765 Publication.)

In use, the LVAD 102 is conventionally operated. Blood enters the LVAD 102 via the inlet 104 and is discharged from the LVAD 102 via the outlet 106 (at a blood outlet pressure). The occluder 108 alternates between an occluded configuration (in which blood cannot pass the occluder 108 and travel in the vascular system 58 downstream from the occluder 108) and flow configuration (in which blood can pass the occluder 108 and travel in the vascular system 58 downstream from the occluder 108). When the occluder 108 is in the occluded configuration, pumped blood being discharged by the LVAD 102 will accumulate upstream of the occluder 108, increasing coronary perfusion. The pressure of the discharged pumped blood will also increase beyond the LVAD blood outlet pressure, via elastic deformation of the coronary vasculature. When the occluder 108 is in the flow configuration, accumulated pump blood will pass the occluder 108 at this higher pressure. The elastically deformed coronary vasculature will return towards is normal state. In this way the systolic/diastolic cycle of the heart may be simulated. (Not shown in the figure are the controller, the actuator, power source and wiring.)

### Second Implementation

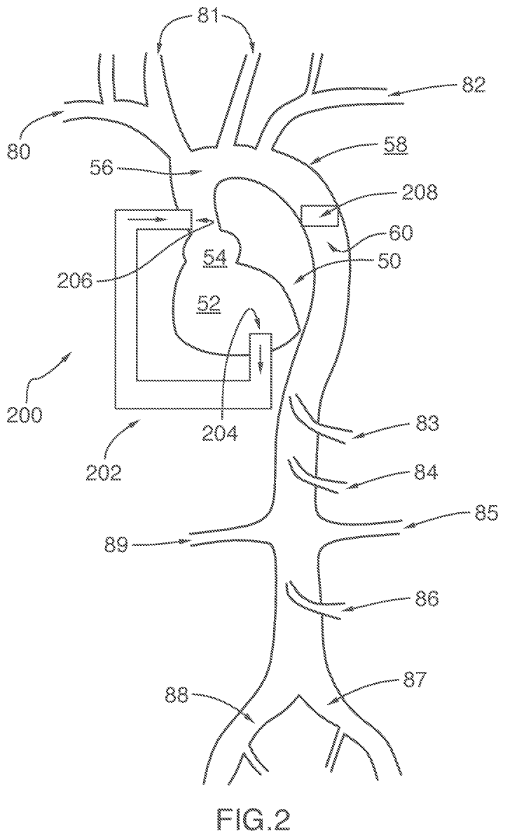

Referring to FIG. 2, there is a shown a second implementation of the present technology 200. In this implementation there is a single fluid pump 202, being a conventional left ventricle assist device (LVAD) (an “apical pump”), which has been surgically implanted using conventional techniques. The LVAD 202 has a blood flow inlet 204 surgically fluidly connected to the left ventricle 52 of the heart 50. The left auricle 54 of the heart 50 is also shown in FIG. 2. The LVAD 202 has a blood flow outlet 206 surgically fluidly connected to the ascending aorta 56. A single occluder 208 (the structure of which is discussed hereinbelow) has been surgically transcatheterly implanted in the proximal descending aorta 60.

In use, the LVAD 202 is conventionally operated. Blood enters the LVAD 202 via the inlet 204 and is discharged from the LVAD 202 via the outlet 206 (at a blood outlet pressure). The occluder 208 alternates between an occluded configuration (in which blood cannot pass the occluder 208 and travel in the vascular system 58 downstream from the occluder 208) and flow configuration (in which blood can pass the occluder 208 and travel in the vascular system 58 downstream from the occluder 208). When the occluder 208 is in the occluded configuration, pumped blood being discharged by the LVAD 202 will accumulate upstream of the occluder 208, increasing carotid artery and coronary perfusion. The pressure of the discharged pumped blood will also increase beyond the LVAD blood outlet pressure, via elastic deformation of the portions of the vascular system 58 fluidly between the outlet 206 and the occluder 208. When the occluder 208 is in the flow configuration, accumulated pump blood will pass the occluder 208 at this higher pressure. The elastically deformed portions of the vasculature will return towards their normal state. In this way the systolic/diastolic cycle of the heart may be simulated. (Not shown in the figure are the controller, the actuator, power source and wiring.)

### Third Implementation

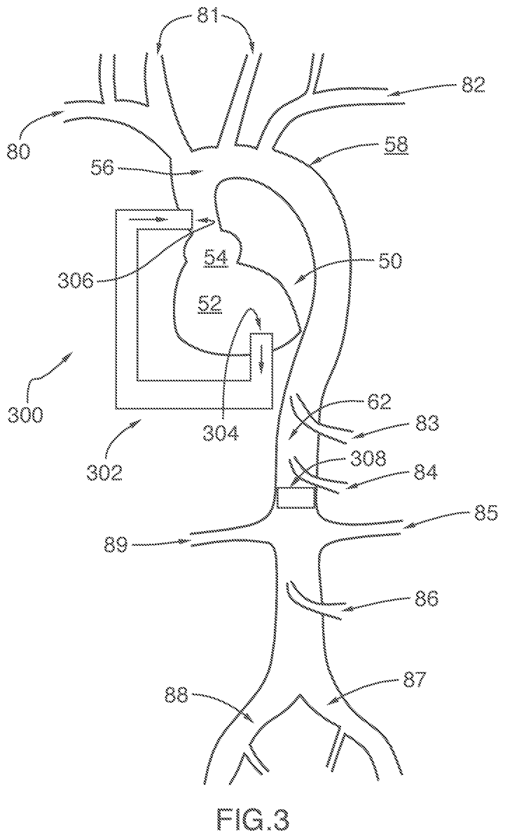

Referring to FIG. 3, there is a shown a third implementation of the present technology 300. In this implementation there is a single fluid pump 302, being a conventional left ventricle assist device (LVAD) (an “apical pump”), which has been surgically implanted using conventional techniques. The LVAD 302 has a blood flow inlet 304 surgically fluidly connected to the left ventricle 52 of the heart 50. The left auricle 54 of the heart 50 is also shown in FIG. 3. The LVAD 302 has a blood flow outlet 306 surgically fluidly connected to the ascending aorta 56. A single occluder 308 (the structure of which is discussed hereinbelow) has been surgically transcatheterly implanted in the descending aorta in a supra-renal position 62.

In use, the LVAD 302 is conventionally operated. Blood enters the LVAD 302 via the inlet 304 and is discharged from the LVAD 302 via the outlet 306 (at a blood outlet pressure). The occluder 308 alternates between an occluded configuration (in which blood cannot pass the occluder 308 and travel in the vascular system 58 downstream from the occluder 308) and flow configuration (in which blood can pass the occluder 308 and travel in the vascular system 58 downstream from the occluder 308). When the occluder 308 is in the occluded configuration, pumped blood being discharged by the LVAD 302 will accumulate upstream of the occluder 308, increasing visceral artery, carotid artery and coronary perfusion. The pressure of the discharged pumped blood will also increase beyond the LVAD blood outlet pressure, via elastic deformation of the portions of the vascular system 58 fluidly between the outlet 306 and the occluder 308. When the occluder 308 is in the flow configuration, accumulated pump blood will pass the occluder 308 at this higher pressure. The elastically deformed portions of the vasculature will return towards their normal state. In this way the systolic/diastolic cycle of the heart may be simulated. (Not shown in the figure are the controller, the actuator, power source and wiring.)

### Fourth Implementation

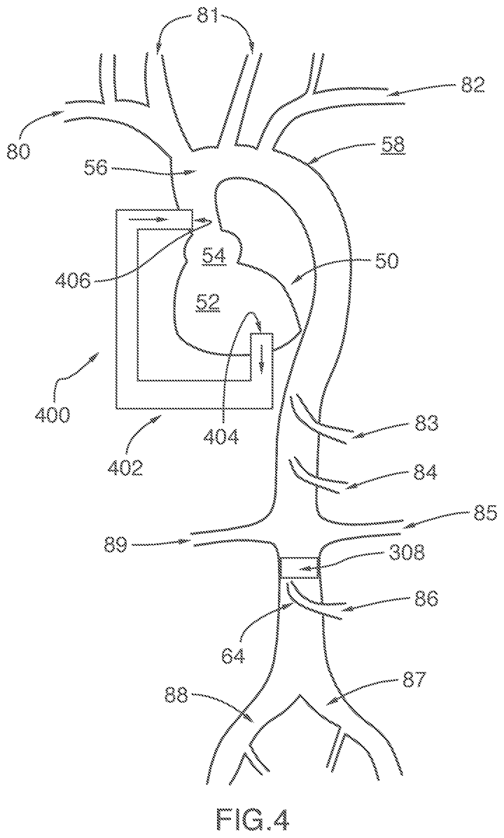

Referring to FIG. 4, there is a shown a fourth implementation of the present technology 400. In this implementation there is a single fluid pump 402, being a conventional left ventricle assist device (LVAD) (an “apical pump”), which has been surgically implanted using conventional techniques. The LVAD 402 has a blood flow inlet 404 surgically fluidly connected to the left ventricle 52 of the heart 50. The left auricle 54 of the heart 50 is also shown in FIG. 4. The LVAD 402 has a blood flow outlet 406 surgically fluidly connected to the ascending aorta 56. A single occluder 408 (the structure of which is discussed hereinbelow) has been surgically transcatheterly implanted in the descending aorta in an infra-renal position 64.

In use, the LVAD 402 is conventionally operated. Blood enters the LVAD 402 via the inlet 404 and is discharged from the LVAD 402 via the outlet 406 (at a blood outlet pressure). The occluder 408 alternates between an occluded configuration (in which blood cannot pass the occluder 408 and travel in the vascular system 58 downstream from the occluder 408) and flow configuration (in which blood can pass the occluder 408 and travel in the vascular system 58 downstream from the occluder 408). When the occluder 408 is in the occluded configuration, pumped blood being discharged by the LVAD 402 will accumulate upstream of the occluder 408, increasing kidney perfusion. The pressure of the discharged pumped blood will also increase beyond the LVAD blood outlet pressure, via elastic deformation of the portions of the vascular system 58 fluidly between the outlet 406 and the occluder 408. When the occluder 408 is in the flow configuration, accumulated pump blood will pass the occluder 408 at this higher pressure. The elastically deformed portions of the vasculature will return towards their normal state. In this way the systolic/diastolic cycle of the heart may be simulated. (Not shown in the figure are the controller, the actuator, power source and wiring.)

### Fifth Implementation

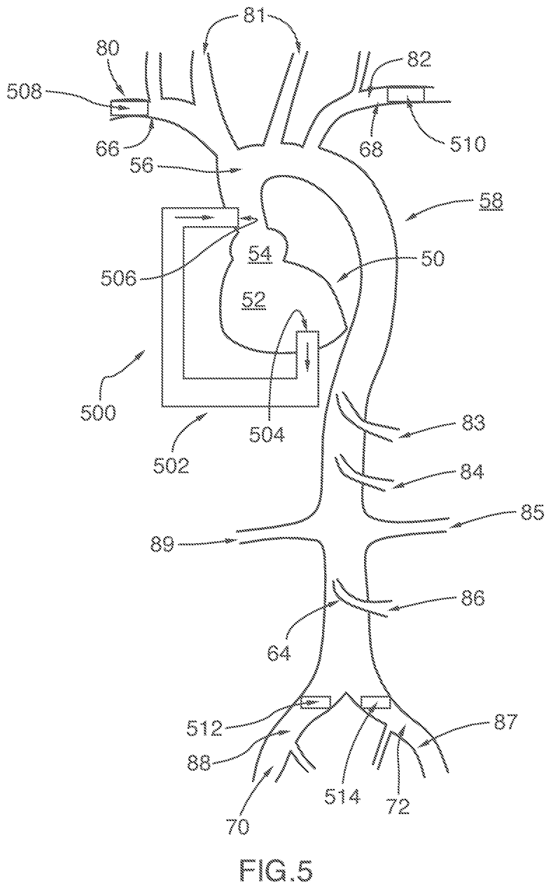

Referring to FIG. 5, there is a shown a fifth implementation of the present technology 500. In this implementation there is a single fluid pump 502, being a conventional left ventricle assist device (LVAD) (an “apical pump”), which has been surgically implanted using conventional techniques. The LVAD 402 has a blood flow inlet 504 surgically fluidly connected to the left ventricle 52 of the heart 50. The left auricle 54 of the heart 50 is also shown in FIG. 5. The LVAD 502 has a blood flow outlet 506 surgically fluidly connected to the ascending aorta 56. In this implementation, multiple occluders 508, 510, 512, 514 (the structure of which is discussed hereinbelow) have been surgically transcatheterly implanted in the vascular system 58. A first occluder 508 has been positioned in the right axillary artery 66. A second occluder 510 has been positioned in the left axillary artery 68. A third occluder 512 has been positioned in the right common iliac artery 70. A fourth occluder 514 has been positioned in the left common iliac artery 72.

In use, the LVAD 502 is conventionally operated. Blood enters the LVAD 502 via the inlet 504 and is discharged from the LVAD 502 via the outlet 506 (at a blood outlet pressure). The occluders 508, 510, 512, 514 can alternate between an occluded configuration (in which blood cannot pass an occluder 508, 510, 512, 514 and travel in the vascular system 58 downstream from that occluder 508, 510, 512, 514) and flow configuration (in which blood can pass an occluder 508, 510, 512, 514 and travel in the vascular system 58 downstream from that occluder 508, 510, 512, 514). When all of the occluders 508, 510, 512, 514 are in the occluded configuration, pumped blood being discharged by the LVAD 402 will accumulate upstream of the occluders 508, 510, 512, 514 increasing carotid and visceral artery perfusion. The pressure of the discharged pumped blood will also increase beyond the LVAD blood outlet pressure, via elastic deformation of the portions of the vascular system 58 fluidly between the outlet 406 and the occluders 508, 510, 512, 514. When an occluder 508, 510, 512, 514 is in the flow configuration, accumulated pump blood will pass that occluder 508, 510, 512, 514 at this higher pressure. The elastically deformed portions of the vasculature will return towards their normal state. In this way the systolic/diastolic cycle of the heart may be simulated. It should be noted that all of the occluders 508, 510, 512, 514 can be in the flow configuration simultaneously; more than one but less than all of the occluders 508, 510, 512, 514 can be in the flow configuration simultaneously; or only one of the occluders 508, 510, 512, 514 can be in the flow configuration at a time. No particular sequence of occluder (or devices) 508, 510, 512, 514 being the flow configuration is required in the context of the present technology. All potential sequences are possible, depending on the intended purpose of the system.

### Sixth Implementation

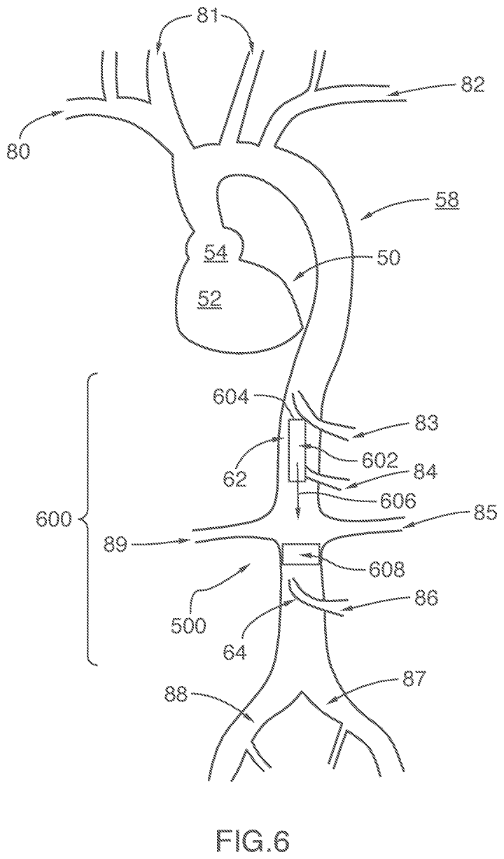

Referring to FIG. 6, there is a shown a sixth implementation of the present technology 600. In this implementation there is a single intra-aortic fluid pump 602, being of the type described in the WO '765 application. The intra-aortic fluid pump 602 has been surgically transcatheterly implanted in the descending aorta in a supra-renal position, as described in the WO '765 Publication. The intra-aortic fluid pump 602 has an upstream blood flow inlet 604 within the descending aorta and a downstream blood flow outlet 606 also within the descending aorta. The left ventricle 52 and auricle 54 of the heart 50 are also shown in FIG. 6.

A single occluder 608 (the structure of which is discussed hereinbelow) has been surgically transcatheterly implanted in the descending aorta in an infra-renal position 64.

In use, the intra-aortic fluid pump 602 is operated as described in the WO '765 Publication. Blood enters the intra-aortic fluid pump 602 via the inlet 604 and is discharged from the intra-aortic fluid pump 602 via the outlet 606 (at a blood outlet pressure). The occluder 608 alternates between an occluded configuration (in which blood cannot pass the occluder 608 and travel in the vascular system 58 downstream from the occluder 608) and flow configuration (in which blood can pass the occluder 608 and travel in the vascular system 58 downstream from the occluder 608). When the occluder 608 is in the occluded configuration, pumped blood being discharged by the intra-aortic fluid pump 602 will accumulate upstream of the occluder 608, increasing renal artery perfusion pressures or pulse pressures. The pressure of the discharged pumped blood will also increase beyond the intra-aortic fluid pump 602 blood outlet pressure, via elastic deformation of the portions of the vascular system 58 fluidly between the outlet 606 and the occluder 608. When the occluder 608 is in the flow configuration, accumulated pump blood will pass the occluder 608 at this higher pressure. The elastically deformed portions of the vasculature will return towards their normal state. In this way the systolic/diastolic cycle of the heart may be simulated. (Not shown in the figure are the controller, the actuator, power source and wiring.)

### Seventh Implementation

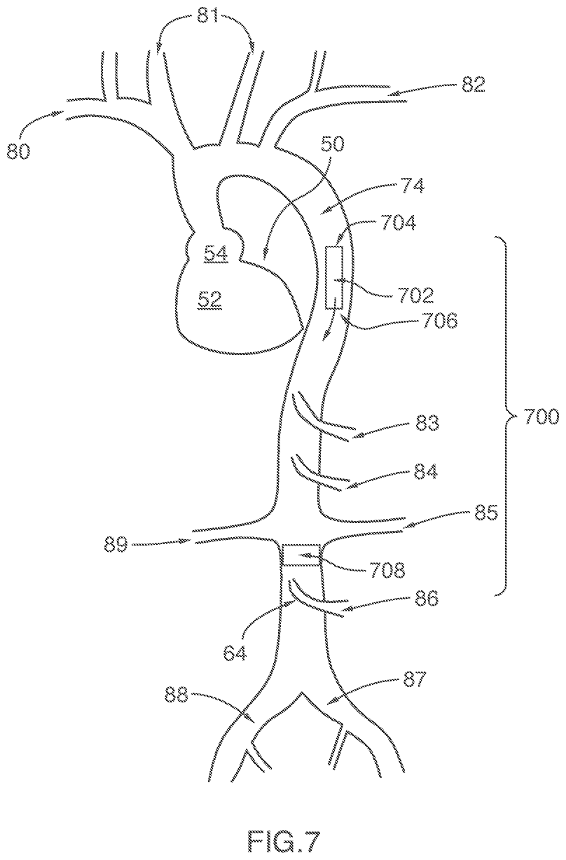

Referring to FIG. 7, there is a shown a seventh implementation of the present technology 700. In this implementation there is a single intra-aortic fluid pump 702, being of the type described in the WO '765 Publication. The intra-aortic fluid pump 702 has been surgically transcatheterly implanted in the descending aorta in a position 74 above the coeliac trunk as described in the WO '765 Publication. The intra-aortic fluid pump 702 has an upstream blood flow inlet 704 within the descending aorta and a downstream blood flow outlet 706 also within the descending aorta. The left ventricle 52 and auricle 54 of the heart 50 are also shown in FIG. 7.

A single occluder 708 (the structure of which is discussed hereinbelow) has been surgically transcatheterly implanted in the descending aorta below in a position the superior mesenteric artery.

In use, the intra-aortic fluid pump 702 is operated as described in the WO '765 Publication. Blood enters the intra-aortic fluid pump 702 via the inlet 704 and is discharged from the intra-aortic fluid pump 702 via the outlet 706 (at a blood outlet pressure). The occluder 708 alternates between an occluded configuration (in which blood cannot pass the occluder 708 and travel in the vascular system 58 downstream from the occluder 708) and flow configuration (in which blood can pass the occluder 708 and travel in the vascular system 58 downstream from the occluder 708). When the occluder 708 is in the occluded configuration, pumped blood being discharged by the intra-aortic fluid pump 702 will accumulate upstream of the occluder 708, increasing splanchnic artery perfusion pressures or pulse pressures. The pressure of the discharged pumped blood will also increase beyond the intra-aortic fluid pump 702 blood outlet pressure, via elastic deformation of the portions of the vascular system 58 fluidly between the outlet 706 and the occluder 708. When the occluder 708 is in the flow configuration, accumulated pump blood will pass the occluder 708 at this higher pressure. The elastically deformed portions of the vasculature will return towards their normal state. In this way the systolic / diastolic cycle of the heart may be simulated. (Not shown in the figure are the controller, the actuator, power source and wiring.)

### Eighth Implementation

Referring to FIG. 8, there is a shown an eighth implementation of the present technology 800. In this implementation there is a single intra-caval fluid pump 802, being of the type described in the WO '765 Publication. The intra-caval fluid pump 802 has been surgically implanted in the inferior vena cava in a position 76 above the renal veins as described in the WO '765 Publication. The intra-caval fluid pump 802 has an upstream blood flow inlet 804 within the inferior vena cava and a downstream blood flow outlet 806 also within the inferior vena cava. The heart 50 is also shown in FIG. 8.

A single occluder 808 (the structure of which is discussed hereinbelow) has been surgically transcatheterly implanted in the superior vena cava in a position 78.

In use, the intra-caval fluid pump 802 is operated as described in the WO '765 Publication. Blood enters the intra-caval fluid pump 802 via the inlet 804 and is discharged from the intra-caval fluid pump 802 via the outlet 806 (at a blood outlet pressure). The occluder 808 alternates between an occluded configuration (in which blood cannot pass the occluder 808 and travel in the vascular system 58 downstream from the occluder 808) and flow configuration (in which blood can pass the occluder 808 and travel in the vascular system 58 downstream from the occluder 808). When the occluder 808 is in the occluded configuration, pumped blood being discharged by the intra-caval fluid pump 802 will accumulate upstream of the occluder 808, increasing right heart filling. (Not shown in the figure are the controller, the actuator, power source and wiring.)

### Occluder—First Embodiment

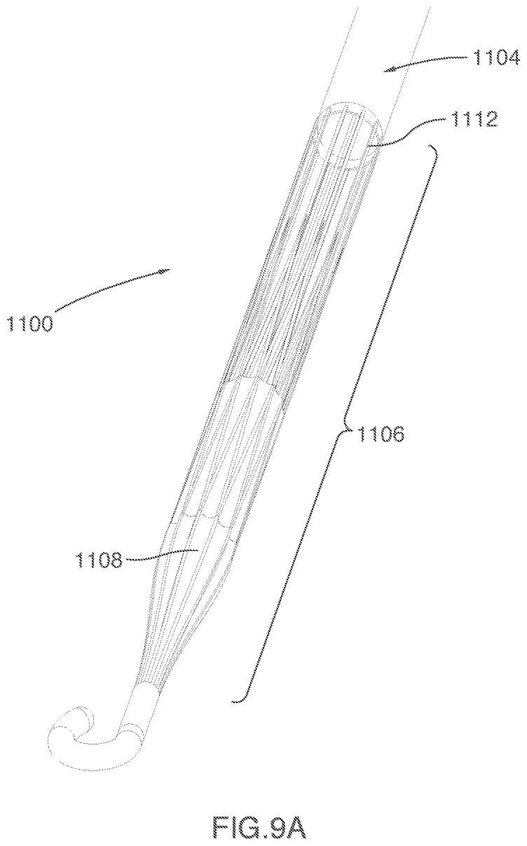

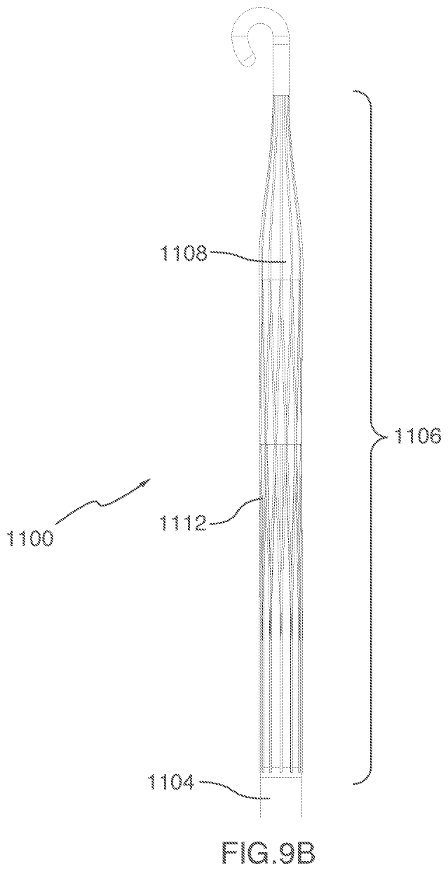

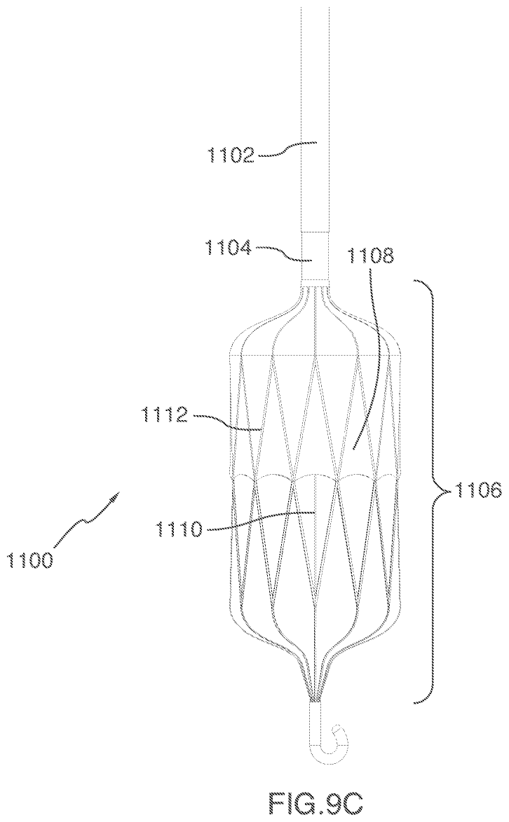

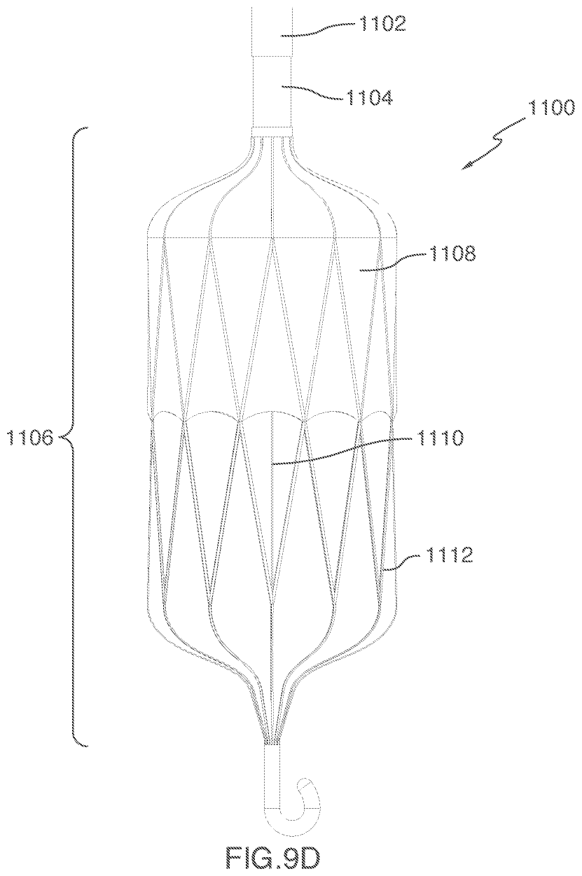

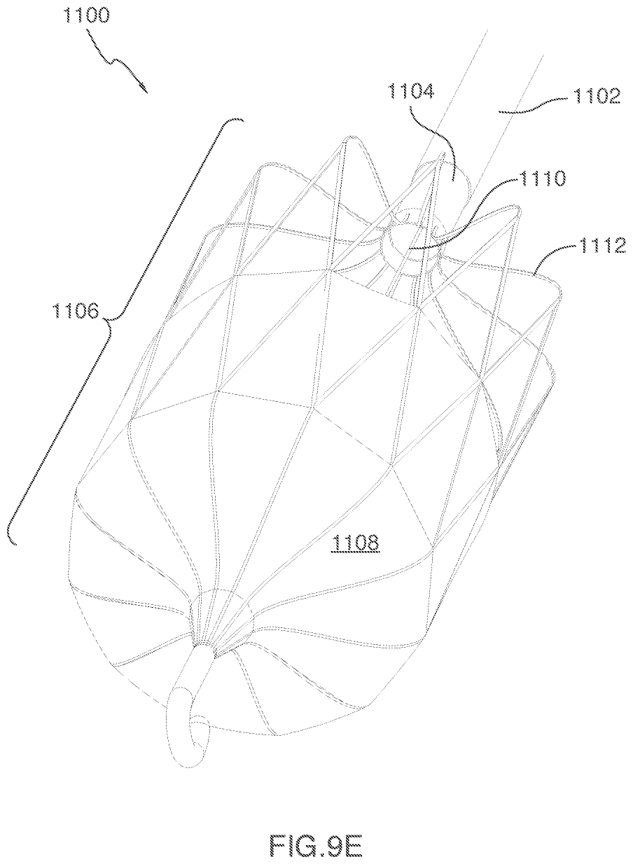

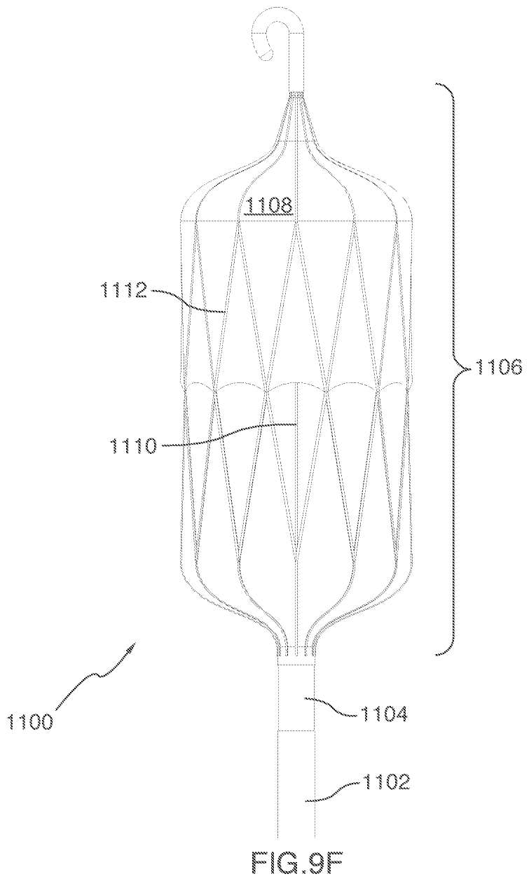

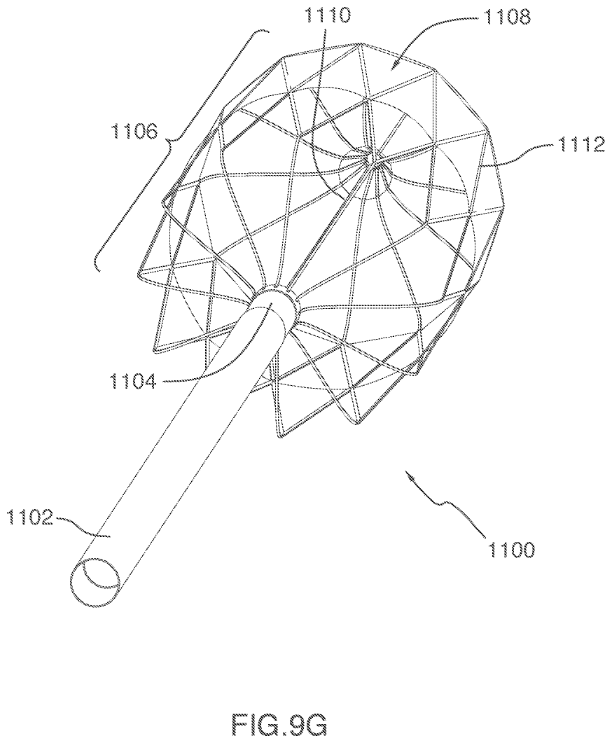

Referring to FIGS. 9A-9G, there is shown a first embodiment of an occluder 1100 of the present technology. As shown in the figures, the device 1100 has two configurations, a closed (flow) configuration and an open (occluded) configuration. The device 1100 is shaped and dimensioned to be implanted within the vascular system of a human body transcatheterly (transcatheter implantations are described in the WO '765 Publication). When the device 1100 is implanted within the vascular system of the human body, and in the closed configuration, the device 1100 is in a flow configuration in which it will allow blood to pass by the device 1100 from upstream of the device 1100 to downstream of the device 1100. When the device 1100 is implanted within the vascular system of the human body at a particular location, the device is dimensioned and shaped such that in the opened configuration the device 1100 is in an occluded configuration. When in the occluded configuration the device 1100 will block blood from passing by the device 1100 from upstream of the device 1100 to downstream of the device 1100.

The device 1100 has an external sheath 1102, an internal guide 1104, an expandable/collapsible cage 1106, an occlusion film 1108, and a control wire 1110. The external sheath 1102 is formed by a catheter. The internal guide 1104 is an elongated tubular structure within the lumen of the external sheath 1102. Within the lumen of the internal guide 1104 is the control wire 1110. Attached to the distal end of the internal guide 1104 is the proximal end of the cage 1106. The cage 1106 is formed of nitinol (a shape-retaining memory alloy). Attached to the distal end of the cage is the control wire 1110. The occlusion film 1108 is connected to the wires 1112 forming the cage 1106. The occlusion film 1108 is made of polytetrafluoroethylene (PTFE), but may be made of any appropriate biocompatible material (e.g., polyester).

In operation, in the closed (flow) configuration the wires 1112 of the cage 1106 are restrained within the external sheath 1102. As the cage 1106 exits the external sheath 1102, the wires 1112 assume their shape, causing the cage 1106 to expand. The cage 1106 will expand to the point where the wires 1112 contact the wall of the blood vessel in which the cage 1106 has been implanted. This will serve to anchor the cage 1106 to the wall. As the cage 1106 has expanded it has extended the occlusion film 1108 that is attached to the wires 1112 of the cage 1106. The occlusion film 1108 is sized and shaped such that it will block the entire lumen of the blood vessel, preventing passage of blood through the blood vessel at that point. At this point the device 1100 is in the open (occluded) configuration. (The process can be analogized to the opening of an umbrella.). Pulling the control wire 1110 will reverse the process, collapsing the cage 1106, returning the device 1100 to the closed (flow) configuration, and allowing blood to once again pass by the device 1100 and flow downstream thereof. (This process can analogized to the closing of an umbrella.)

In this first embodiment there are two sub-embodiments, the first being shown in FIGS. 9A, 9B, 9E, 9F and 9G, while the second is shown in FIGS. 9C and 9D. The difference between these two sub-embodiments is the location of the occlusion film 1108. In the first sub-embodiment, the occlusion film 1108 is positioned at the distal end of the cage 1106 (thus the blood flow would be occluded by the “topside of the umbrella”). In the second sub-embodiment, the occlusion film is positioned at the proximal end of the cage 1108 (thus the blood flow would be occluded by the “underside of the umbrella”).

The control wire is attached to a conventional electromechanical actuation system (not shown, but inside sheath 1102) that includes a conventional microcontroller, a conventional rechargeable battery, and a conventional motor (e.g., a solenoid, a camshaft motor, etc.). Examples of such conventional components are provided in the following documents, the entirety of each of which is incorporated herein by reference:
International Patent Application Publication No. WO 2019/183247 A1, published Sep. 26, 2019, entitled “Circulatory Assist Pump” (Second Heart Assist, Inc.)
International Patent Application Publication No. WO 2019/083989 A1, published May 2, 2019, entitled “Systems and Methods for Selectively Occluded the Superior Vena Cava for Treating Heart Conditions” (Tufts Medical Center, Inc.)
International Patent Application Publication No. WO 2015/109028 A1, published Jul. 23, 2015, entitled “Apparatus and Methods for Optimizing Intra Cardiac Filling Pressures, Heart Rate, and Cardiac Output” (Kaiser et al.)
International Patent Application Publication No. WO 2014/070472 A1, published May 8, 2014, entitled “Leadless Pacemaker System” (Medtronic, Inc.)
International Patent Application Publication No. WO 2012/094641 A2, published Jul. 12, 2012, entitled “Percutaneous Heart Pump” (Thoratec Corp. et al.)
   U.S. Patent Application Publication No. US 2020/0261633 A1, published Aug. 20, 2020, entitled “Intravascular Blood Pump” (ABIOMED EUROPE GmbH)
   U.S. Patent Application Publication No. US 2019/0126014 A1, published May 2, 2019, entitled “Systems and Methods for Selectively Occluded the Superior Vena Cava for Treating Heart Conditions” (Kapur et al.)
   U.S. Patent Application Publication No. US 2009/0247945 A1, published Oct. 1, 2009, entitled “Balloons and Balloon Catheter Systems for Treating Vascular Occlusions” (Levit et al.)
   U.S. Pat. No. 9,808,633 B2, granted Nov. 7, 2017, entitled “Leadless Pacemaker System” (Bonner et al.)
   U.S. Pat. No. 9,375,580 B2, granted Jun. 28, 2016, entitled “Leadless Pacemaker System” (Bonner et al.)
   U.S. Pat. No. 9,216,298 B2, granted Dec. 22, 2015, entitled “Leadless Cardiac Pacemaker System with Conductive Communication” (Jacobson)

The control wire can also be operated manually (if so appropriate) via a conventional mechanical actuation system. See for example. U.S. Pat. No. 10,279,094 B2, grated May 7, 2019, entitled “Endovascular Variable Aortic Control Catheter” (Williams et al.), which is incorporated herein by reference in its entirety.

### Occluder—Second Embodiment

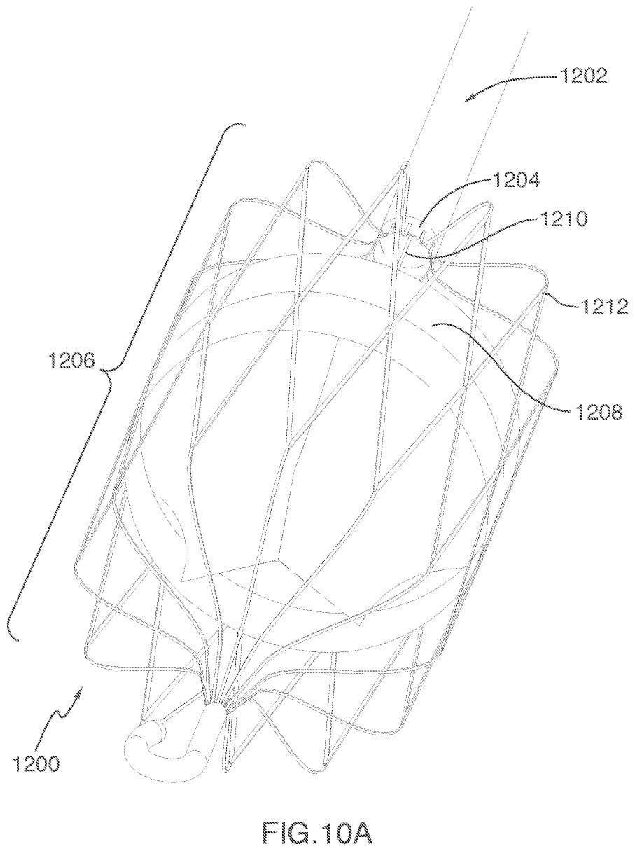

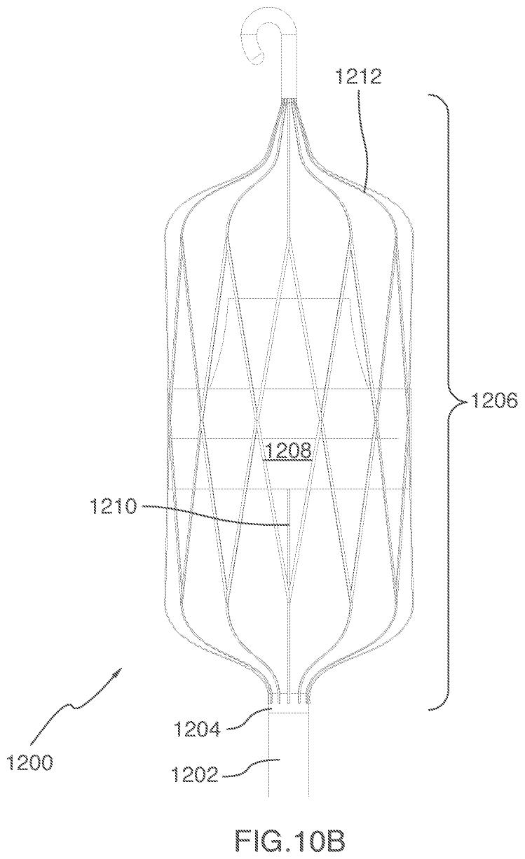

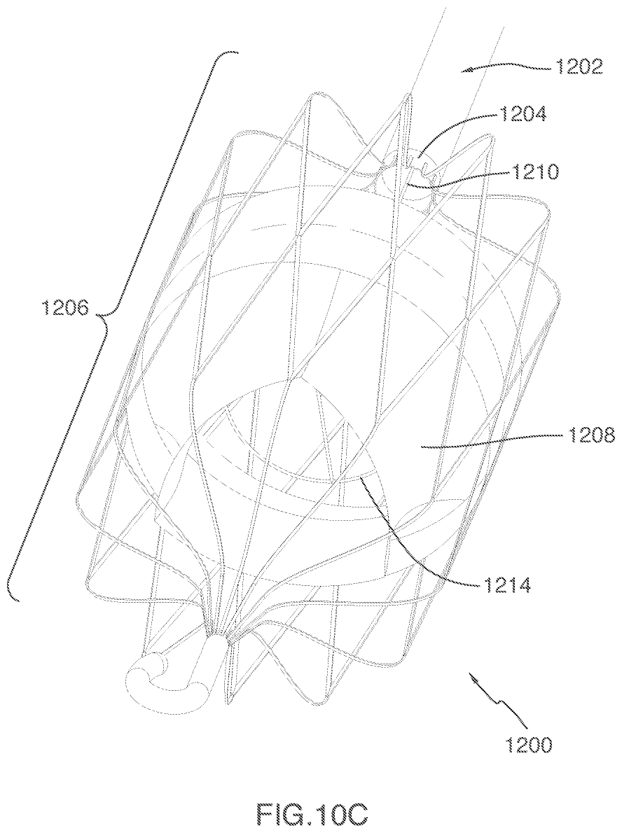

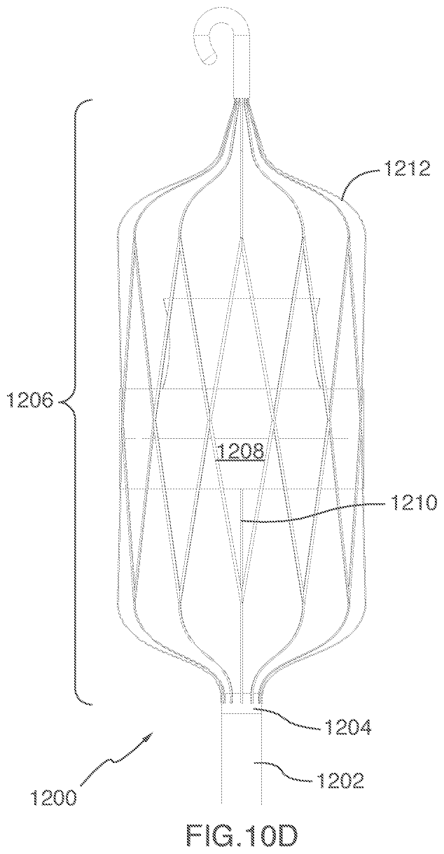

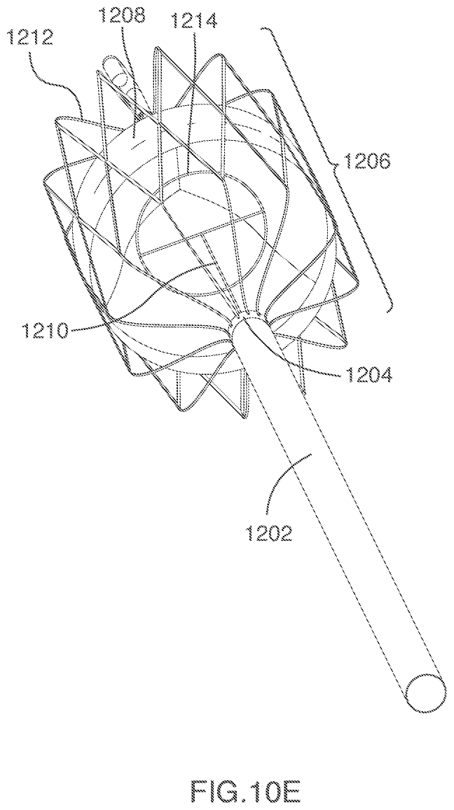

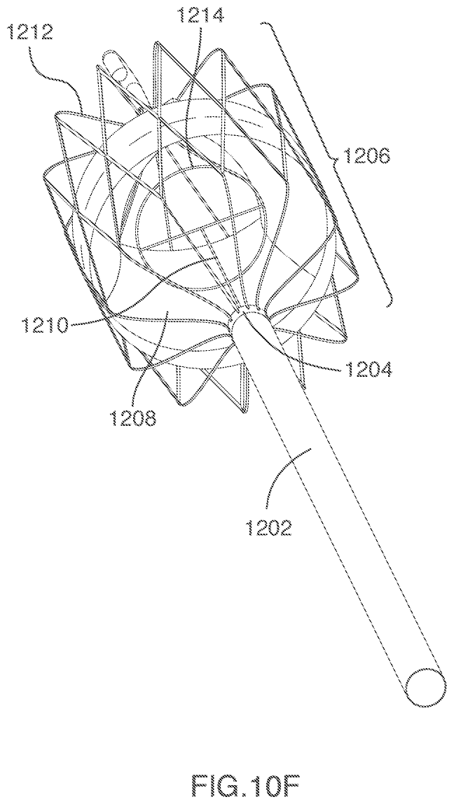

Referring to FIGS. 10A-10F, there is shown a second embodiment of an occluder 1200 of the present technology. As shown in the figures, the device 1200 has two operational configurations, a closed configuration and an open configuration. The device 1200 is shaped and dimensioned to be implanted within the vascular system of a human body transcatheterly. When the device 1200 is implanted within the vascular system of the human body, and in the open configuration, the device 1200 is in a flow configuration in which it will allow blood to passing by the device 1200 from upstream of the device 1200 to downstream of the device 1200. When the device 1200 is implanted within the vascular system of the human body at a particular location, the device is dimensioned and shaped such that in the closed configuration the device 1200 is in an occluded configuration. When in the occluded configuration the device 1200 will block blood from passing by the device 1200 from upstream of the device 1200 to downstream of the device 1200.

The device 1200 has an external sheath 1202, an internal guide 1204, an expandable/collapsible cage 1206, an occlusion film 1208, and a control wire 1210. The external sheath 1202 is formed by a catheter. The internal guide 1204 is an elongated tubular structure within the lumen of the external sheath 1202. Within the lumen of the internal guide 1204 is the control wire 1210. Attached to the distal end of the internal guide 1204 is the proximal end of the cage 1206. The cage 1206 is formed of nitinol (a shape-retaining memory alloy). Attached to the distal end the control wire 1210 is valve actuating element 1214. The occlusion film 1208 is connected to the wires 1212 forming the cage 1206 at the distal end of the cage. In this embodiment, the occlusion film 1208 forms a leaflet valve, which is actuated by the valve actuating element 1214. In this embodiment the occlusion film 1208 is polytetrafluoroethylene.

In operation, in the delivery configuration the wires 1212 of the cage 1206 are restrained within the external sheath 1202. On implantation, as the cage 1206 exits the external sheath 1202, the wires 1212 assume their shape, causing the cage 1206 to expand. The cage 1206 will expand to the point where the wires 1212 contact the wall of the blood vessel in which the cage 1206 has been implanted. This will serve to anchor the cage 1206 to the wall. As the cage 1206 has expanded it has extended the occlusion film 1208 that is attached to the wires 1212 of the cage 1206. The occlusion film 1208 is sized and shaped such that it will form a leaflet valve when unfurled. When the valve is closed, the film 1208 can block the entire lumen of the blood vessel, preventing passage of blood through the blood vessel at that point. At this point the device 1200 is in the closed (occluded) configuration. When the valve is open, the film 1208 will not block the entire lumen of the blood vessel, allowing blood to pass by the device 1200 and flow downstream thereof. At this point the device is in the open (flow) configuration.

Moving the control wire 1210 positions the valve actuating element 1214 so as to open or close the valve formed by the film 1208. The control wire 1210 may be moved by any conventional system as described hereinabove with respect to the first embodiment.

### Occluder—Third Embodiment

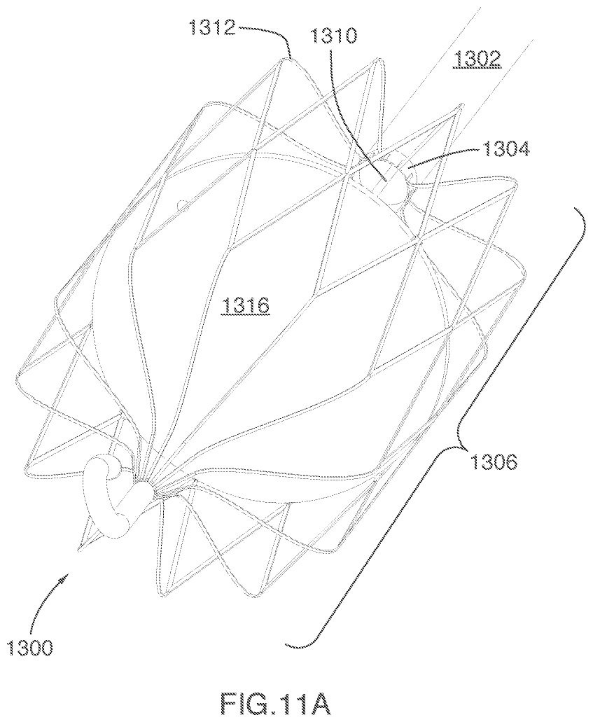

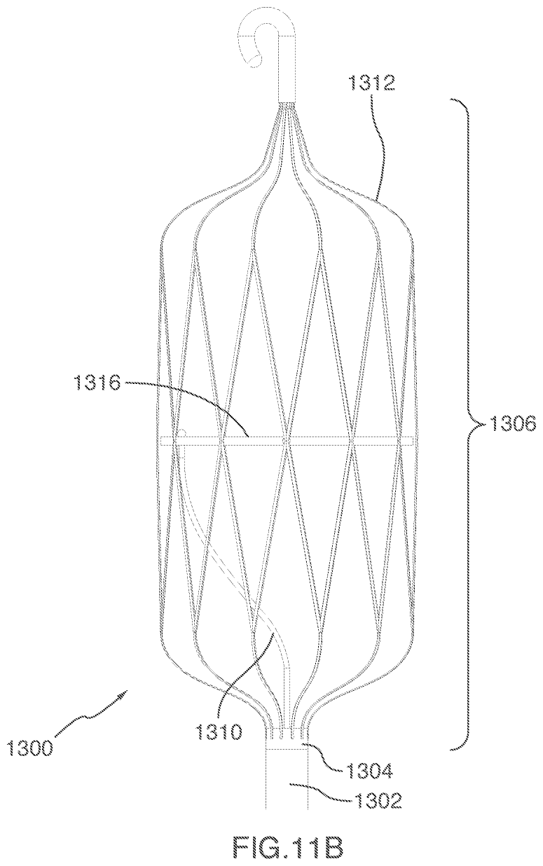

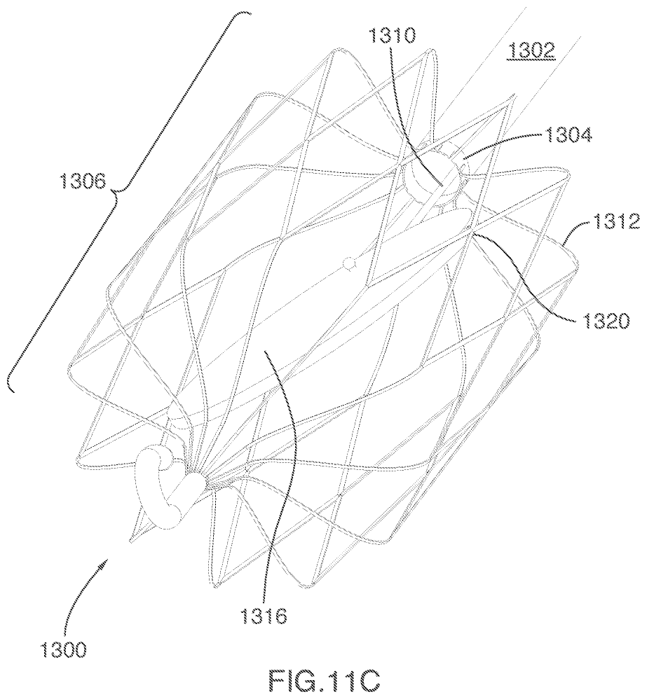

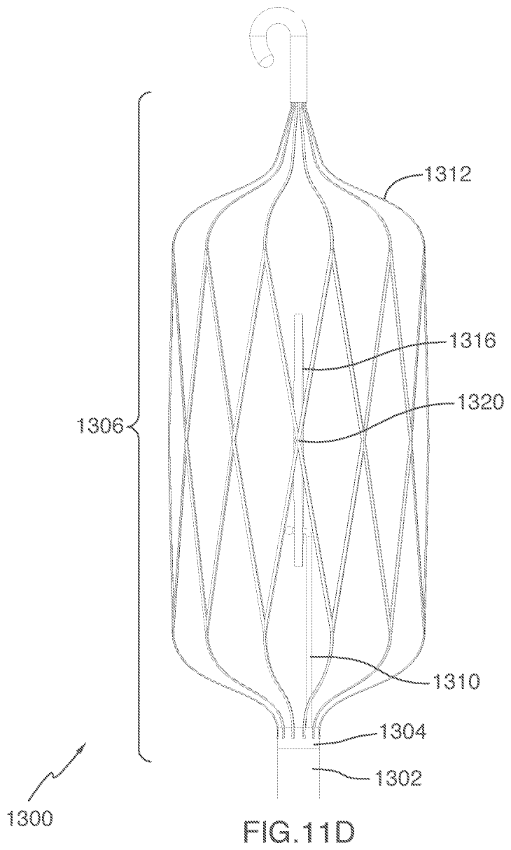

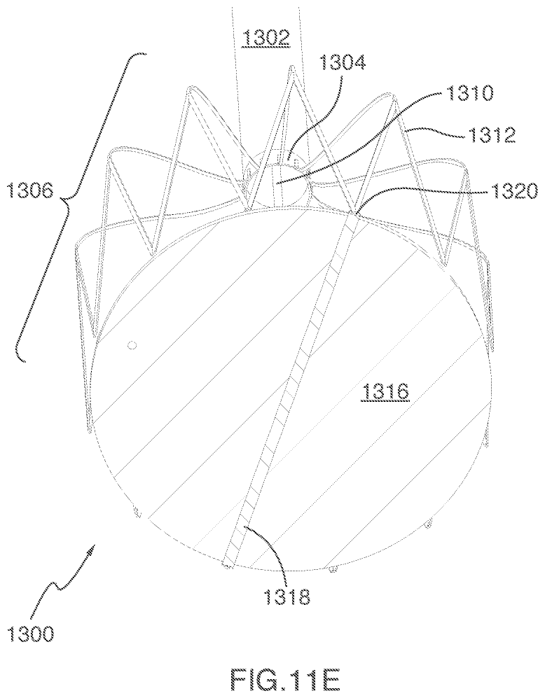

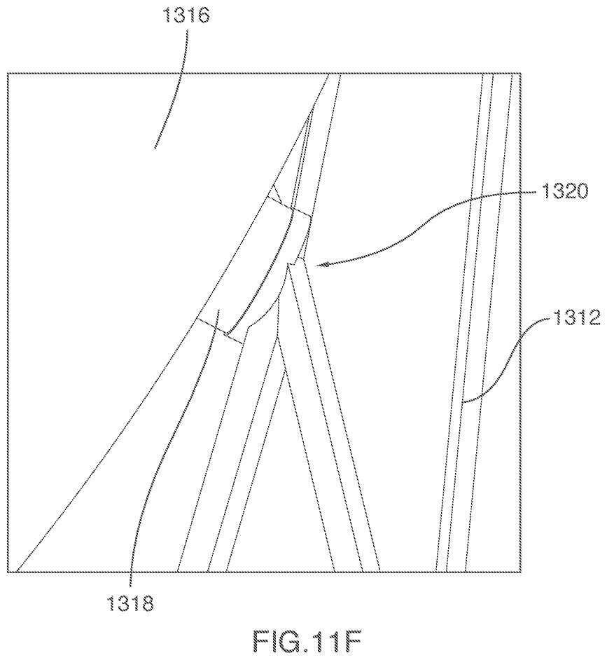

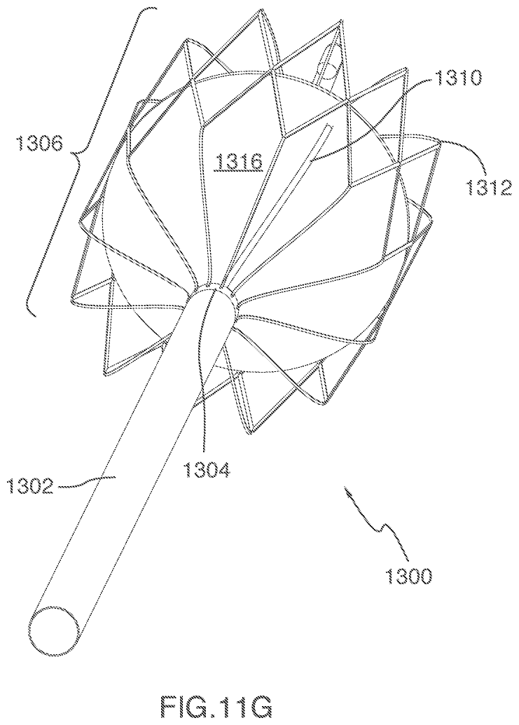

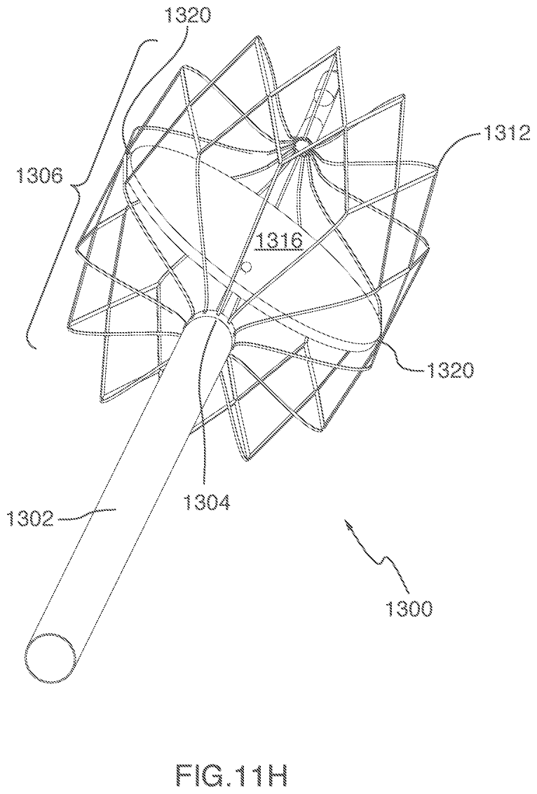

Referring to FIGS. 11A-11H, there is shown a third embodiment of an occluder 1300 of the present technology. As shown in the figures, the device 1300 has two operational configurations, a closed configuration and an open configuration. The device 1300 is shaped and dimensioned to be implanted within the vascular system of a human body transcatheterly. When the device 1300 is implanted within the vascular system of the human body, and in the open configuration, the device 1300 is in a flow configuration in which it will allow blood to passing by the device 1300 from upstream of the device 1300 to downstream of the device 1300. When the device 1300 is implanted within the vascular system of the human body at a particular location, the device is dimensioned and shaped such that in the closed configuration the device 1300 is in an occluded configuration. When in the occluded configuration the device 1300 will block blood from passing by the device 1300 from upstream of the device 1300 to downstream of the device 1300.

The device 1300 has an external sheath 1302, an internal guide 1304, an expandable/collapsible cage 1306, an occluding element 1316, and a control wire 1310. The external sheath 1302 is formed by a catheter. The internal guide 1304 is an elongated tubular structure within the lumen of the external sheath 1302. Within the lumen of the internal guide 1304 is the control wire 1310. Attached to the distal end of the internal guide 1304 is the proximal end of the cage 1306. The cage 1306 is formed of nitinol (a shape-retaining memory alloy). The occluding element 1316 is attached to the distal end the control wire 1310. The occluding element 1316 is connected to the wires 1312 forming the cage 1306 via a pivoting element 1318. In this embodiment, the occluding element 1316 forms a butterfly valve, which is actuated (to pivot) by the control wire 1310. The occluding element 1316 and the pivoting element 1318 are made of nitinol.

In operation, in the delivery configuration the wires 1312 of the cage 1306 are restrained within the external sheath 1306. On implantation, as the cage 1306 exits the external sheath 1306, the wires 1312 assume their shape, causing the cage 1306 to expand. The cage 1306 will expand to the point where the wires 1312 contact the wall of the blood vessel in which the cage 1306 has been implanted. This will serve to anchor the cage 1306 to the wall. As the cage 1306 has allowed the occluded element 1316 to expand as well. The occluded element 1316 is sized and shaped and attached to the pivoting element 1318, which itself is pivotably attached to the cage 1306 (at pivot point 1320, see e.g., FIG. 11F) such that it will form a butterfly valve when expanded. When the valve is closed, the element 1316 can block the entire lumen of the blood vessel, preventing passage of blood through the blood vessel at that point. At this point the device 1300 is in the closed (occluded) configuration. When the valve is open, the element 1308 will not block the entire lumen of the blood vessel, allowing blood to pass by the device 1300 and flow downstream thereof. At this point the device is in the open (flow) configuration.

Moving the control wire 1310 pivots element 1316 so as to open or close the valve formed by the element 1316. The control wire 1210 may be moved by any conventional system as described hereinabove with respect to the first embodiment.

### Miscellaneous

The present technology is not limited in its application to the details of construction and the arrangement of components set forth in the previous description or illustrated in the drawings. The disclosure is capable of other embodiments, implementations and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the previous description, the same numerical references refer to similar elements.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two 10 specified features or components with or without the other. For example, “A and/or B” is to be taken as specific disclosure of each of (i) A, (ii) B and (iii) A and B, just as if each is set out individually herein.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1-91. (canceled)

92. An assist system for assisting blood flow in a vascular system, the assist system comprising:
an implantable pump having a fluid inlet and a fluid outlet, each of the fluid inlet and the fluid outlet configured for being in fluid communication with the vascular system;
an occluder element configured for being implanted in the vascular system downstream of the fluid outlet of the implantable pump,
the occluder element having an occluded configuration in which the occluder element occludes the vascular system so that blood flowing downstream in the vascular system from the fluid outlet of the implantable pump toward the occluder element is at least partially blocked from flowing further downstream past the occluder element, and
the occluder element having a flow configuration in which blood flowing downstream in the vascular system from the fluid outlet of the implantable pump toward the occluder element is capable of flowing further downstream past the occluder element; and
an actuator element operatively connected to the occluder element for actuating the occluder element between the occluded configuration and the flow configuration.

93. The assist system according to claim 92, wherein the implantable pump, the occluder element, and the actuator element together form a unitary structure.

94. The assist system according to claim 93, wherein the unitary structure is an implantable unitary structure in which the actuator element comprises an end portion configured for connecting to an extracorporeal controller for actuating the occluder element between the occluded configuration and the flow configuration.

95. The assist system according to claim 92, wherein the implantable pump is an intravascular pump.

96. The assist system according to claim 95, wherein the intravascular pump comprises an anchor configured for anchoring the intravascular pump within the vascular system.

97. The assist system according to claim 92, wherein the occluder element comprises a cage configured for anchoring the assist system within the vascular system.

98. The assist system according to claim 97, wherein the cage is overcomeably biased toward the occluded configuration.

99. The assist system according to claim 92, wherein the occluder element comprises a cage and an occlusion film connected to the cage, and the actuator element comprises a control wire operatively connected to the cage for actuating the occluder element between the occluded configuration and the flow configuration.

100. The assist system according to claim 99, wherein the occlusion film is connected to a proximal end portion of the cage.

101. The assist system according to claim 99, wherein the occlusion film is connected to a distal end portion of the cage.

102. The assist system according to claim 92, wherein the occluder element comprises a cage and an occlusion film connected to the cage, the occlusion film forming a valve, and the actuator element comprises a control wire and a valve actuating element connected to the control wire, the valve actuating element configured for actuating the valve between the occluded configuration and the flow configuration.

103. The assist system according to claim 102, wherein the valve is a leaflet valve that is at least partially disposed within the cage.

104. The assist system according to claim 92, wherein the occluder element comprises a cage and a butterfly valve pivotably connected to the cage, and the actuator element comprises a control wire operatively connected to the butterfly valve for actuating the butterfly valve between the occluded configuration and the flow configuration.

105. The assist system according to claim 92, wherein the occluder element comprises a balloon.

106. The assist system according to claim 92, wherein the occluder element comprises a first occluder operatively connected to the actuator element for actuating the first occluder between the occluded configuration and the flow configuration, and a second occluder operatively connected to the actuator element for actuating the second occluder between the occluded configuration and the flow configuration.

107. The assist system according to claim 106, wherein the first occluder and the second occluder are actuatable in the flow configuration independently.

108. The assist system according to claim 106, wherein the first occluder and the second occluder are actuatable in the flow configuration simultaneously.

109. The assist system according to claim 106, wherein the first occluder and the second occluder are actuatable in the flow configuration sequentially.

110. The assist system according to claim 106, wherein the actuator element comprises a first actuator operatively connected to the first occluder for actuating the first occluder between the occluded configuration and the flow configuration, and a second actuator operatively connected to the second occluder for actuating the second occluder between the occluded configuration and the flow configuration.
